SEQUENCE LISTING (26 sequences)

SEQ ID NO: 1 - protein, 975 aa, Artificial Sequence
  Synthetic Polypeptide
MSAEREIPAEDSIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGNARTTIVICCSPASFNESETKSTLDFGRRAKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMSSHQKKRITEMLTNLLRDLGEVGQAIAPGESSIDLKMSALAGTDASKVEEDFTMARLFISKMKTEAKNIAQRCSNMETQQADSNKKISEYEKDLGEYRLLISQHEARMKSLQESMREAENKKRTLEEQIDSLREECAKLKAAEHVSAVNAEEKQRAEELRSMFDSQMDELREAHTRQVSELRDEIAAKQHEMDEMKDVHQKLLLAHQQMTADYEKVRQEDAEKSSELQNIILTNERREQARKDLKGLEDTVAKELQTLHNLRKLFVQDLQQRIRKNVVNEESEEDGGSLAQKQKISFLENNLDQLTKVHKQLVRDNADLRCELPKLEKRLRCTMERVKALETALKEAKEGAMRDRKRYQYEVDRIKEAVRQKHLGRRGPQAQIAKPIRSGQGAIAIRGGGAVGGPSPLAQVNPVNS

SEQ ID NO: 2 - protein, 503 aa, Artificial Sequence
  Synthetic Polypeptide
MSAEREIPAEDSIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGNARTTIVICCSPASFNESETKSTLDFGRRAKTVKNVVCVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 3 - protein, 503 aa, Artificial Sequence
  Synthetic Polypeptide
MSAKKKEEKGKNIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGNARTTIVICCSPASFNESETKSTLDFGRRAKTVKNVVCVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 4 - protein, 503 aa, Artificial Sequence
  Synthetic Polypeptide
MSAEREIPAEDSIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGNARTTIVICCSPASFNESETKSTLDFGRRAKTILNKPEVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 5 - protein, 503 aa, Artificial Sequence
  Synthetic Polypeptide
MSAEREIPAEDSIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGRTRTTIVICCSPASFNESETKSTLDFGRRAKTVKNVVCVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 6 - protein, 503 aa, Artificial Sequence
  Synthetic Polypeptide
MSAKKKEEKGKNIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGNARTTIVICCSPASFNESETKSTLDFGRRAKTILNKPEVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 7 - protein, 503 aa, Artificial Sequence
  Synthetic Polypeptide
MSAKKKEEKGKNIKVVCRFRPLNDSEEKAGSKFVVKFPNNVEENCISIAGKVYLFDKVFKPNASQEKVYNEAAKSIVTDVLAGYNGTIFAYGQTSSGKTHTMEGVIGDSVKQGIIPRIVNDIFNHIYAMEVNLEFHIKVSYYEIYMDKIRDLLDVSKVNLSVHEDKNRVPYVKGATERFVSSPEDVFEVIEEGKSNRHIAVTNMNEHSSRSHSVFLINVKQENLENQKKLSGKLYLVDLAGSEKVSKTGAEGTVLDEAKNINKSLSALGNVISALADGNKTHIPYRDSKLTRILQESLGGRTRTTIVICCSPASFNESETKSTLDFGRRAKTILNKPEVNEELTAEEWKRRYEKEKEKNARLKGKVEKLEIELARWRAGETVKAEEQINMEDLMEASTPNLRKAMEAPAAAEISGHIVRSPMVGTFYRTPSPDAKAFIEVGQKVNVGDTLCIVEAMKMMNQIEADKSGTVKAILVESGQPVEFDEPLVVIELSETSGHHHHHH

SEQ ID NO: 8 - protein, 9 aa, Artificial Sequence
  Synthetic Polypeptide
MADLAECNI

SEQ ID NO: 9 - protein, 13 aa, Artificial Sequence
  Synthetic Polypeptide
MSAEREIPAEDSI

SEQ ID NO: 10 - protein, 19 aa, Artificial Sequence
  Synthetic Polypeptide
MASQPNSSAKKKEEKGKNI

SEQ ID NO: 11 - protein, 12 aa, Artificial Sequence
  Synthetic Polypeptide
IKNTVCVNVELT

SEQ ID NO: 12 - protein, 12 aa, Artificial Sequence
  Synthetic Polypeptide
VKNVVCVNEELT

SEQ ID NO: 13 - protein, 10 aa, Artificial Sequence
  Synthetic Polypeptide
ILNKPEVNQK

SEQ ID NO: 14 - protein, 6 aa, Artificial Sequence
  Synthetic Polypeptide
LGGNCR

SEQ ID NO: 15 - protein, 6 aa, Artificial Sequence
  Synthetic Polypeptide
LGGNAR

SEQ ID NO: 16 - protein, 6 aa, Artificial Sequence
  Synthetic Polypeptide
LGGRTR

SEQ ID NO: 17 - protein, 13 aa, Artificial Sequence
  Synthetic Polypeptide
MSAKKKEEKGKNI

SEQ ID NO: 18 - protein, 6 aa, Artificial Sequence
  Synthetic Polypeptide
LGGRTR

SEQ ID NO: 19 - protein, 12 aa, Artificial Sequence
  Synthetic Polypeptide
ILNKPEVNEELT

SEQ ID NO: 20 - protein, 1056 aa, Homo Sapiens
MASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNILNKPEVNQKLTKKALIKEYTEEIERLKRDLAAAREKNGVYISEENFRVMSGKLTVQEEQIVELIEKIGAVEEELNRVTELFMDNKNELDQCKSDLQNKTQELETTQKHLQETKLQLVKEEYITSALESTEEKLHDAASKLLNTVEETTKDVSGLHSKLDRKKAVDQHNAEAQDIFGKNLNSLFNNMEELIKDGSSKQKAMLEVHKTLFGNLLSSSVSALDTITTVALGSLTSIPENVSTHVSQIFNMILKEQSLAAESKTVLQELINVLKTDLLSSLEMILSPTVVSILKINSQLKHIFKTSLTVADKIEDQKKELDGFLSILCNNLHELQENTICSLVESQKQCGNLTEDLKTIKQTHSQELCKLMNLWTERFCALEEKCENIQKPLSSVQENIQQKSKDIVNKMTFHSQKFCADSDGFSQELRNFNQEGTKLVEESVKHSDKLNGNLEKISQETEQRCESLNTRTVYFSEQWVSSLNEREQELHNLLEVVSQCCEASSSDITEKSDGRKAAHEKQHNIFLDQMTIDEDKLIAQNLELNETIKIGLTKLNCFLEQDLKLDIPTGTTPQRKSYLYPSTLVRTEPREHLLDQLKRKQPELLMMLNCSENNKEETIPDVDVEEAVLGQYTEEPLSQEPSVDAGVDCSSIGGVPFFQHKKSHGKDKENRGINTLERSKVEETTEHLVTKSRLPLRAQINL

SEQ ID NO: 21 - protein, 9 aa, Artificial Sequence
  Synthetic Polypeptide
PAEDSIGGC

SEQ ID NO: 22 - protein, 14 aa, Artificial Sequence
  Synthetic Polypeptide
MSAEREIPAEDSIC

SEQ ID NO: 23 - protein, 19 aa, Artificial Sequence
  Synthetic Polypeptide
MASQPNSSAKKKEEKGKNI

SEQ ID NO: 24 - protein, 6 aa, Artificial Sequence
  Synthetic Polypeptide
EKGKNI

SEQ ID NO: 25 - protein, 6 aa, Artificial Sequence
  Synthetic Polypeptide
PAEDSI

SEQ ID NO: 26 - protein, 13 aa, Artificial Sequence
  Synthetic Polypeptide
MSAEREIPAEDSI

(19) United States
(12) Patent Application Publication
(10) Pub. No.: US 2014/0057285 A1
(43) Pub. Date: Feb. 27, 2014

(54) COMPOSITIONS AND METHODS TARGETING FORCE GENERATION IN KINESIN

(71) Applicants: Matthew J. Lang, Nashville, TN (US); William Hesse, Lebanon, NJ (US); Wonmuk Hwang, College Station, TX (US); Martin Karplus, Cambridge, MA (US)

(72) Inventors: Matthew J. Lang, Nashville, TN (US); William Hesse, Lebanon, NJ (US); Wonmuk Hwang, College Station, TX (US); Martin Karplus, Cambridge, MA (US)

(21) Appl. No.: 14/003,070

(22) PCT Filed: Mar. 5, 2012
(86) PCT No.: PCT/US12/27773

§ 371 Date: Oct. 8, 2013

Related U.S. Application Data
(60) Provisional application No. 61/449,670, filed on Mar. 5, 2011.

(51) Int. Cl. C12N 9/14; G01N 33/573; C12Q 1/34

(52) U.S. Cl. CPC C12N 9/14; C12Q 1/34; G01N 33/573
USPC 435/7.4; 435/195; 435/18

(57) ABSTRACT

In some aspects, the invention provides chimeric kinesin proteins. In other aspects the invention provides nucleic acids encoding chimeric kinesin proteins. Compositions and kits are provided that comprise chimeric kinesin proteins and nucleic acids encoding the same. Antibodies and antigen binding fragments that selectively bind kinesin proteins are also provided.

### RELATED APPLICATIONS

The present application claims priority under 35 U.S.C. §119(e) to U.S. provisional application, U.S. Ser. No. 61/449,670 filed Mar. 5, 2011 the entire contents of which are incorporated herein by reference.

### FIELD OF THE INVENTION

The invention relates to kinesin proteins and nucleic acids encoding kinesin proteins.

### BACKGROUND OF INVENTION

Kinesin proteins are a class of motor proteins found in eukaryotic cells. Kinesin proteins move along microtubules powered by the hydrolysis of ATP. It has been found that microtubule-based molecular motors, such as kinesins, have roles in various cellular functions, including cell division. Inhibition of kinesin motor function provides a unique strategy for developing targeted therapeutics. In particular, inhibition of kinesin motor function provides a strategy for developing targeted anti-cancer agents, as exemplified by the Kinesin-5 inhibitor, monastrol.

### SUMMARY OF INVENTION

According to some aspects of the invention, chimeric kinesin proteins are provided. These chimeric kinesin proteins comprise one or more regions from at least two different kinesin proteins. Chimeric kinesin proteins have a variety of uses. For example, chimeric kinesin proteins are useful for examining molecular mechanisms of kinesin function (e.g., motor action, force generation, etc.). As another example, chimeric proteins are useful for identifying agents (e.g., antibodies, small molecules, peptides, etc.) that alter the function of kinesin proteins.

In some embodiments, chimeric kinesin proteins comprise one or more regions having an amino acid sequence of a first kinesin protein and (i.) a coverstrand having an amino acid sequence of a coverstrand of second kinesin protein, or (ii.) a necklinker having an amino acid sequence of a necklinker of a second kinesin protein, or (iii.) an L13 region having an amino acid sequence of an L13 region of a second kinesin protein, in which the first kinesin protein is different than the second kinesin protein. The first kinesin protein and second kinesin protein may each be selected from the group consisting of Kinesin-1, Kinesin-2, Kinesin-3, Kinesin-4, Kinesin-5, Kinesin-6, Kinesin-7, Kinesin-8, Kinesin-9, Kinesin-10, Kinesin-11, Kinesin-12, Kinesin-13, and Kinesin-14 proteins. In some embodiments, the chimeric kinesin proteins comprise one or more regions having an amino acid sequence of a kinesin protein that is not a Kinesin-5 protein and one or more regions having an amino acid sequence of a Kinesin-5 protein. In some embodiments, the chimeric kinesin proteins have one or more regions having an amino acid sequence of a kinesin protein that is a Kinesin-1 protein and one or more regions having an amino acid sequence of a Kinesin-5 protein.

According to some aspects of the invention, nucleic acids encoding any of the chimeric kinesin proteins disclosed herein are provided. Expression vectors that have a promoter operably linked to a nucleic acid encoding a chimeric kinesin protein are provided in other aspects of the invention. Cells harboring the nucleic acids or expression vectors are also provided. According to some aspects of the invention, compositions or kits are provided that comprise any of the chimeric kinesin proteins, nucleic acids, expression vectors and cells disclosed herein.

According to some aspects of the invention, methods are provided for characterizing the ability of test agents to affect motility of a kinesin protein. The methods typically involve the use of a chimeric kinesin protein to identify test agents that target particular regions of a kinesin protein.

According to some aspects of the invention, an antibody or antigen binding fragment thereof is provided that binds selectively to an amino acid sequence of a kinesin protein. According to some aspects of the invention, an antibody or antigen binding fragment thereof is provided that binds selectively to an amino acid sequence of a coverstrand, necklinker, or L13 region of a kinesin protein. According to some aspects of the invention, an antibody or antigen binding fragment thereof is provided that binds selectively to an amino acid sequence of PAEDSI (SEQ ID NO: 25) or MSAEREIPAEDSI (SEQ ID NO: 26). According to some aspects of the invention, an antibody or antigen binding fragment thereof is provided that binds selectively to an amino acid sequence of MSAKKKEEKGKNI (SEQ ID NO: 17), MASQPNSSAKKKEEKGKNI (SEQ ID NO: 23) or EKGKNI (SEQ ID NO: 24).

In some embodiments, compositions or kits are provided that comprise any of the antibodies or antigen binding fragments, or polyclonal antibody preparations disclosed herein. In some embodiments, a cell line is provided that produces any of the antibodies or antigen binding fragments disclosed herein.

### BRIEF DESCRIPTION OF DRAWINGS

FIG. 1: Structural alignment of Kinesin-1 and Kinesin-5 (Eg5).

FIG. 2A: Comparison of kinesin protein regions.

FIG. 2B: Alignment of certain chimeric kinesin proteins sequences.

FIG. 3: Representative traces of runs from chimeric kinesin proteins used herein

FIG. 4: Stall forces of chimeric kinesin proteins used in this study.

FIG. 5: The force-velocity behavior of chimeric kinesin proteins used in this study.

FIG. 6: The distributions of velocity at stall from the stall force data.

FIG. 7: Unloaded velocities and run lengths for each of the constructs used in this study.

FIG. 8: Western blot used for determination of bleeds to use for purification.

FIG. 9: Western blot showing the specificity of the antibodies to the Kinesin-1 coverstrand of D. melanogaster.

FIG. 10: Antibody titration curve, which shows the disruption of Kinesin-1's motility as a function of the concentration of antibody.

FIG. 11A: Antibody titration curve, which shows the disruption of Kinesin-5's motility as a function of the concentration of antibody.

FIG. 11B: Results of a gliding filament assay showing the effects of a polyclonal antibody preparation directed against the Kinesin-5 cover strand on Kinesin-5's motility.

### DEFINITIONS

As used herein, the term “agents” or “test agents” refers to peptides, polypeptides, proteins, peptide and/or nucleic acid aptamers, small molecules, organic and/or inorganic compounds, polysaccharides, lipids, nucleic acids, particles, antibodies, ligands, or combinations thereof.

As used herein, the term “antibody fragment” refers to any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

As used herein, the term “antibody” refers to an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in the present invention.

As used herein, the term “chimeric kinesin protein” refers to a kinesin protein composed of regions from at least two different kinesin proteins. In some embodiments, a chimeric kinesin protein is a kinesin protein (e.g., a Kinesin-1 protein) for which the coverstrand region is substituted (e.g., by recombinant DNA techniques) with the coverstrand region of a different kinesin protein (e.g., a Kinesin-5 protein). In some embodiments, a chimeric kinesin protein is a kinesin protein (e.g., a Kinesin-1 protein) for which the necklinker region is substituted with the necklinker region of a different kinesin protein (e.g., a Kinesin-5 protein). In some embodiments, a chimeric kinesin protein is a kinesin protein (e.g., a Kinesin-1 protein) for which the L13 region is substituted with the L13 region of a different kinesin protein (e.g., a Kinesin-5 protein).

As used herein, the term “diabodies” refers to dimeric scFvs. The components of diabodies typically have shorter peptide linkers than most scFvs, and they show a preference for associating as dimers.

As used herein, the term “F(ab′)₂ fragment” refers to an antibody fragment essentially equivalent to that obtained from immunoglobulins (typically IgG) by digestion with an enzyme pepsin at pH 4.0-4.5. The fragment may be recombinantly produced.

As used herein, the term “Fab fragment” refers to an antibody fragment essentially equivalent to that obtained by digestion of immunoglobulins (typically IgG) with the enzyme papain. The Fab fragment may be recombinantly produced. The heavy chain segment of the Fab fragment is the Fd piece.

As used herein, the term “Fab′ fragment” is an antibody fragment essentially equivalent to that obtained by reduction of the disulfide bridge or bridges joining the two heavy chain pieces in the F(ab′)₂ fragment. The Fab′ fragment may be recombinantly produced.

As used herein, the term “Fv fragment” refers to an antibody fragment which consists of one V_{H} and one V_{L} domain held together by noncovalent interactions. The term “dsFv” is used herein to refer to an Fv with an engineered intermolecular disulfide bond to stabilize the V_{H}-V_{L} pair.

As used herein, the term “kinesin protein” refers to a protein comprising at least one domain having homology to a kinesin motor domain. Kinesin proteins typically have ATP binding activity, microtubulin binding activity and/or microtubule-based motor activity. Kinesin proteins typically have a heavy chain that may be composed of multiple structural domains. The heavy chain may be composed of a large globular N-terminal domain which is responsible for the motor activity of kinesin, a central alpha-helical coiled-coil domain that mediates the heavy chain dimerization, and a small globular C-terminal domain which interacts with other proteins (such as the kinesin light chains), vesicles and membranous organelles. A kinesin protein may have any of the following signature domains: Gene3D: G3DSA:3.40.850.10; Pfam (PF00225); PRINTS: PRO0380; PROSITE profile: PS50067; and SMART: SM00129. A kinesin protein may be any kinesin protein identified in Lawrence C. J., et al., A standardized kinesin nomenclature JCB vol. 167 no. 1 19-22, Oct. 11, 2004, the contents of which are incorporated herein by reference. A kinesin protein may be a Kinesin-1, Kinesin-2, Kinesin-3, Kinesin-4, Kinesin-5, Kinesin-6, Kinesin-7, Kinesin-8, Kinesin-9, Kinesin-10, Kinesin-11, Kinesin-12, Kinesin-13, or Kinesin-14 protein. The Kinesin-5 protein may be Eg5, which has a sequence as set forth in SEQ ID NO: 20.

As used herein, the term “nucleic acid” refers to the phosphate ester form of ribonucleotides (RNA molecules) or deoxyribonucleotides (DNA molecules), or any phosphodiester analogs, in either single-stranded form, or a double-stranded helix. Double-stranded DNA-DNA, DNA-RNA and RNA-RNA helices are possible. The term nucleic acid, and in particular DNA or RNA, refers to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA).

As used herein, the term “protein” comprises a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins preferably contain only natural amino acids, although non-natural amino acids and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these.

As used herein, the term “single-chain Fvs (scFvs)” refers to recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

As used herein, the term “small molecule” is used to refer to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. Preferred small molecules are biologically active in that they produce a biological effect in animals, preferably mammals, more preferably humans.

### DETAILED DESCRIPTION OF CERTAIN EMBODIMENTS OF THE INVENTION

Chimeric kinesin proteins are provided herein that comprise one or more regions from at least two different kinesin proteins. Chimeric kinesin proteins may be used for examining molecular mechanisms of kinesin function (e.g., motor action, force generation, etc.) and for identifying agents at affect kinesin motor function. For example, to investigate the relative roles of the coverstrand, β9, and L13 in motor behavior chimeric KHC/Eg5 constructs were created that incorporate the sequences for certain elements from Eg5 into the KHC motorhead. It has been determined that stall force and unloaded run length are affected by the substitution of Eg5 structural elements into KHC. These results indicate that the motors operate well with a matched Cover Neck Bundle (CNB) and that L13 strongly affects the mechanical strength of the motor. While a matched CNB appears to make the relative motor function more robust, β9 has a larger impact on motor function than β0. Furthermore, these structural elements cause the motor to stall at lower forces, be slower, and less processive, but they alone do not turn KHC into Eg5.

Chimeric kinesin proteins may comprise one or more regions having an amino acid sequence of a first kinesin protein and (i.) a coverstrand having an amino acid sequence of a coverstrand of second kinesin protein, or (ii.) a necklinker having an amino acid sequence of a necklinker of a second kinesin protein, or (iii.) an L13 region having an amino acid sequence of an L13 region of a second kinesin protein, in which the first kinesin protein is different than the second kinesin protein. The first kinesin protein and second kinesin protein may each be selected from the group consisting of Kinesin-1, Kinesin-2, Kinesin-3, Kinesin-4, Kinesin-5, Kinesin-6, Kinesin-7, Kinesin-8, Kinesin-9, Kinesin-10, Kinesin-11, Kinesin-12, Kinesin-13, and Kinesin-14.

The chimeric kinesin proteins may comprise one or more regions having an amino acid sequence of a kinesin protein that is not a Kinesin-5 protein and one or more regions having an amino acid sequence of a Kinesin-5 protein. In some embodiments, the chimeric kinesin proteins have one or more regions having an amino acid sequence of a kinesin protein that is a Kinesin-1 protein and one or more regions having an amino acid sequence of a Kinesin-5 protein.

The chimeric kinesin protein may comprise a coverstrand having an amino acid sequence of a coverstrand of a Kinesin-5 protein; or a necklinker having an amino acid sequence of a necklinker of a Kinesin-5 protein; or an L13 region having an amino acid sequence of an L13 region of a Kinesin-5 protein.

The chimeric kinesin protein may comprise one or more regions of a human kinesin protein. The chimeric kinesin protein may comprise one or more regions of a kinesin protein from a species selected from the group consisting of: Arabidopsis thaliana; Aspergillus nidulans; Bombyx mori; Candida albicans; Caenorhabditis elegans; Chlamydomonas rheinhardtii; Cricetulus griseus; Cyanophora paradoxa; Cylindrotheca fusiformis; Danio rerio; Dictyostelium discoideum; Drosophila melanogaster; Drosophila yakuba; Gallus gallus; Homo Sapiens; Leishmania chagasi; Leishmania major; Loligo pealii; Lymantria dispar; Monodelphis domestica; Morone saxatilis; Mus musculus; Nectria haematococca; Neurospora crassa; Nicotiana tabacum; Oryza sativa; Paracentrotus lividus; Plasmodium falciparum; Rattus norvegicus; Saccharomyces cerevisiae; Schizosaccharomyces pombe; Solanum tuberosum; Strongylocentrotus purpuratus; Syncephalastrum racemosum; Tetrahymena thermophila; Trypanosoma brucei; Ustilago maydis; Volvox carteri; and Xenopus laevis. The kinesin protein may be a non-Kinesin-5 protein or a Kinesin-5 protein from any of the foregoing organisms. The non-Kinesin-5 protein may be selected from the group consisting of: Kinesin-1, Kinesin-2, Kinesin-3, Kinesin-4, Kinesin-6, Kinesin-7, Kinesin-8, Kinesin-9, Kinesin-10, Kinesin-11, Kinesin-12, Kinesin-13, and Kinesin-14 proteins. The chimeric kinesin protein may comprise an amino acid sequence as set forth in any of SEQ ID NO: 3 to 7.

Compositions are provided that comprise any of the chimeric kinesin proteins disclosed herein. Often the compositions further comprise buffers, salts, protease inhibitors, and/or other agents suitable for ensuring protein stability and/or function. Compositions comprising reaction components (e.g., buffers comprising ATP, microtubules, etc.) are also provided.

Nucleic acids encoding the chimeric kinesin proteins are also provided. In some cases, expression vectors are provided that comprise a promoter operably linked to a nucleic acid encoding a chimeric kinesin protein. Isolated cells harboring the nucleic acids or expression vectors are also provided. The isolated cells may be any eukaryotic cells, including any mammalian cells (e.g., human cells) or cells of any of the following species: Arabidopsis thaliana; Aspergillus nidulans; Bombyx mori; Candida albicans; Caenorhabditis elegans; Chlamydomonas rheinhardtii; Cricetulus griseus; Cyanophora paradoxa; Cylindrotheca fusiformis; Danio rerio; Dictyostelium discoideum; Drosophila melanogaster; Drosophila yakuba; Gallus gallus; Homo Sapiens; Leishmania chagasi; Leishmania major; Loligo pealii; Lymantria dispar; Monodelphis domestica; Morone saxatilis; Mus musculus; Nectria haematococca; Neurospora crassa; Nicotiana tabacum; Oryza sativa; Paracentrotus lividus; Plasmodium falciparum; Rattus norvegicus; Saccharomyces cerevisiae; Schizosaccharomyces pombe; Solanum tuberosum; Strongylocentrotus purpuratus; Syncephalastrum racemosum; Tetrahymena thermophila; Trypanosoma brucei; Ustilago maydis; Volvox carteri; and Xenopus laevis.

An antibody or antigen binding fragment thereof is provided that binds selectively to an amino acid sequence of a kinesin protein or chimeric kinesin protein. The antibody or antigen binding fragment thereof may bind selectively to an amino acid sequence of a coverstrand, necklinker, or L13 region of a kinesin protein. The antibody or antigen binding fragment thereof may bind selectively to an amino acid sequence of PAEDSI (SEQ ID NO: 25) or MSAEREIPAEDSI (SEQ ID NO: 26). The antibody or antigen binding fragment thereof may bind selectively to an amino acid sequence of MSAKKKEEKGKNI (SEQ ID NO: 17) or MASQPNSSAKKKEEKGKNI (SEQ ID NO: 23). The antibody or antigen binding fragment thereof may bind selectively to an amino acid sequence of EKGKNI (SEQ ID NO: 24). Compositions or kits are provided that comprise any of the antibodies or antigen binding fragments disclosed herein. The antibodies may be polyclonal or monoclonal. Cell lines are provided that produce any of the antibodies or antigen binding fragments disclosed herein (e.g., a hybridoma).

### Methods

Methods are provided herein for characterizing the ability of a test agent to affect motility of a kinesin protein. The methods typically involve the use of a chimeric kinesin protein to identify test agents that target particular regions of a kinesin protein. For example, the methods may involve obtaining a chimeric kinesin protein comprising one or more regions having an amino acid sequence of a first kinesin protein, and one or more regions having an amino acid sequence of a second kinesin protein (in which the first and second kinesin proteins are different); and assessing motility of the chimeric kinesin protein in the presence a test agent. The first and second kinesin proteins may each be selected from: Kinesin-1, Kinesin-2, Kinesin-3, Kinesin-4, Kinesin-6, Kinesin-7, Kinesin-8, Kinesin-9, Kinesin-10, Kinesin-11, Kinesin-12, Kinesin-13, and Kinesin-14 proteins. In some embodiments, the first kinesin protein is Kinesin-1. In some embodiments, the second kinesin protein is Kinesin-5. The one or more regions of the second kinesin protein may, for example, be a coverstrand of second kinesin protein, and/or a necklinker of a second kinesin protein, and/or an L13 region having of an L13 region of a second kinesin protein.

The step of assessing motility of the chimeric kinesin protein in the presence the test agent may involve subjecting the chimeric kinesin protein to a motility assay in the presence of the test agent, in which the results of the motility assay indicate whether the test agent inhibits motility of the chimeric kinesin protein. The assessment step may also involve subjecting the first kinesin protein to a motility assay in the presence the test agent, in which the results of the motility assay indicate whether the test agent inhibits motility of the first kinesin protein. In this context, if the test agent inhibits motility of the chimeric kinesin protein but does not substantially inhibit motility of the first kinesin protein, then the test agent may be identified as targeting the one or more regions of the second kinesin protein. The assessment step may also involve subjecting the second kinesin protein to a motility assay in the presence the test agent, in which the results of the motility assay indicate whether the test agent inhibits motility of the second kinesin protein. In this context, if the test agent inhibits motility of the chimeric kinesin protein and motility of the second kinesin protein, then the test agent may be identified as targeting the one or more regions of the second kinesin protein. Any suitable motility assay for assessing kinesin protein motility may be used, including, for example, a gliding filament assay, a stall force assay, or other suitable method known in the art.

In some embodiments, the chimeric kinesin proteins disclosed herein may be utilized in phage or yeast display technologies (or other similar screening technologies) to identify test agents that are relatively strong binders to a region of interest of kinesin proteins. In some embodiments, a test agent that binds specifically to a cover strand, necklinker or L13 region of a kinesin protein may be identified using a suitable display technology. For example, a library of cells (e.g., yeast cells) may be established that displays variants of a test agent (e.g., an aptamer or antibody). Cells in the library may be contacted with a chimeric kinesin protein having a region of interest (e.g., a coverstrand) of a kinesin protein of interest (e.g., a Kinesin-5 protein). Cells in the library that bind to the chimeric kinesin protein may then be contacted with the kinesin protein of interest (a non-chimera) to enrich in cells that bind specifically to the region of interest. This process may be repeated to further enrich for test agents that are relatively strong binders to a region of interest of kinesin proteins.

In some embodiments, structural models of chimeric kinesin proteins may be used to identify test agents that target particular regions of a kinesin protein by virtual (in silico) screening techniques. For an example of virtual screening methods employed to identify inhibitors of kinesin see: Shanthi Nagarajan, Dimitrios A. Skoufias, Frank Kozielski, and Ae Nim Pae. Receptor—Ligand Interaction-Based Virtual Screening for Novel Eg5/Kinesin Spindle Protein Inhibitors, Journal of Medicinal Chemistry Article ASAP Publication Date (Web): Feb. 6, 2012 (employing structure-based virtual screening of a database of 700,000 compounds to identify three Eg5 inhibitors bearing quinazoline or thioxoimidazolidine scaffolds.)

### Kits

The chimeric kinesin proteins (or nucleic acids encoding the same, or antibodies or antigen binding fragments that bind selectively to the same) described herein may, in some embodiments, be provided in kits to facilitate their use in assays, research or other applications. A kit may include one or more containers housing the components of the invention and instructions for use. Specifically, such kits may include one or more chimeric kinesin proteins (or nucleic acids encoding the same, or antibodies or antigen binding fragments that bind selectively to the same) described herein, along with instructions describing the intended application and the proper use of these components. The kits typically comprise a container (e.g., a vial, a tube, a multi-well plate, a package, etc.) housing any of the chimeric kinesin proteins, nucleic acids encoding the same, or any of the compositions disclosed herein.

Exemplary embodiments of the disclosure will be described in more detail by the following examples. These embodiments are exemplary of the disclosure, which one skilled in art will recognize is not limited to the exemplary embodiments.

### EXAMPLES

### Example 1

### Study of Chimeric Kinesin Constructs

Chimeric Kinesin-1 (KHC)/Kinesin-5 (Eg5) constructs were developed. The constructs were used to study the force generation mechanism of the motor protein. The kinesin family of proteins walk along microtubules to carry cargo or pull microtubules along each other and do so by hydrolyzing a single ATP per 8 nm step. The results disclosed herein indicate that the kinesin's force generation mechanism of the Cover Neck Bundle (CNB) utilizes the formation of a β-sheet between the coverstrand and β9 and subsequent folding of this sheet towards the motor head.

Experiments conducted using dimeric forms of Eg5 (a member of the Kinesin-5 family) have shown that the motor is capable of generating nearly analogous amounts of force as Kinesin-1, but that it dissociates from the microtubule under load rather than coming to a true stall. Chimeras employing the Eg5 CNB, and in some cases L13 mutated into the KHC motorhead, were developed. The chimeras were used to further study the CNB model and to determine if a higher stall force kinesin could be generated. It was found that motors with a matched CNB performed the significantly well.

These results disclosed herein indicate that the CNB mechanism may only be a partial explanation of force generation for Eg5, and that L13 may influence the amount of force that is able to be extracted from the CNB. These results show that the parts of kinesin are not directly interchangeable, and that aspects of Eg5's force generation mechanism may be distinct compared with that of Kinesin-1.

Chimeras Used in this Study

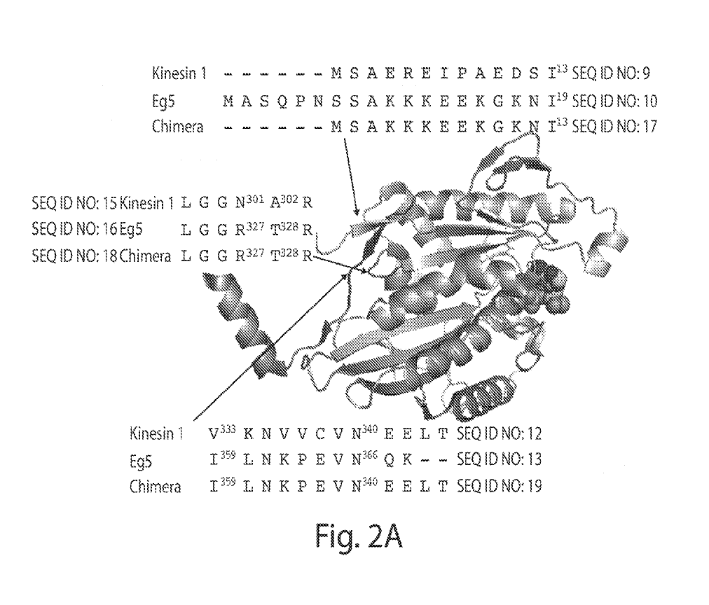

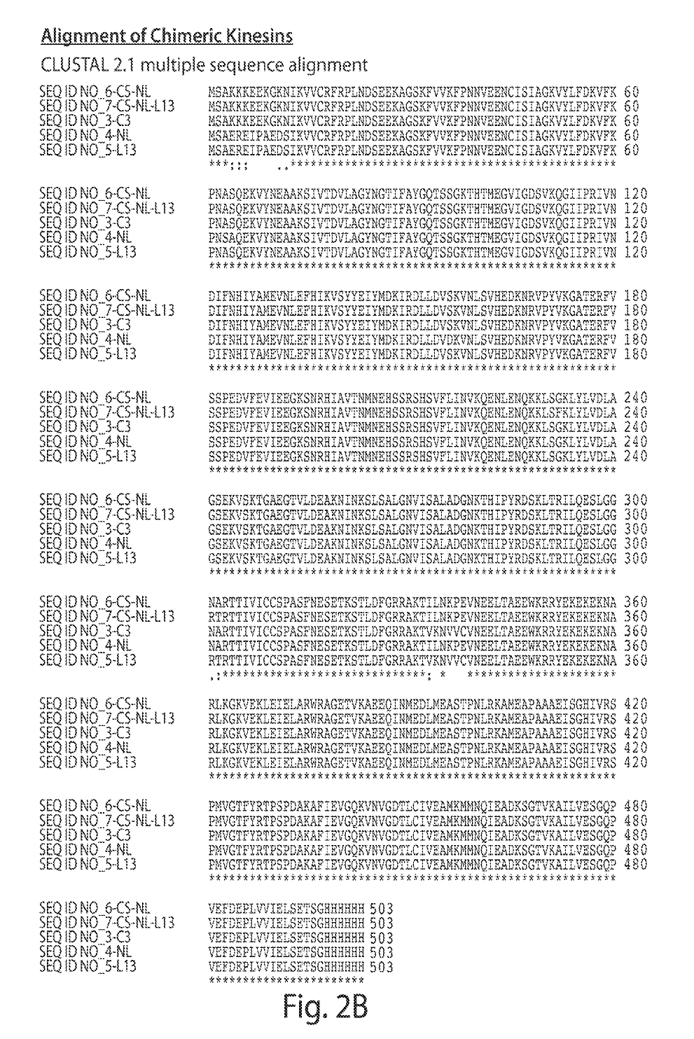

Chimeras of Kinesin-1 and Kinesin-5 have been used to elucidate the relative significance of the various structural elements in the kinesin motor head. Examples of studies conducted with Kinesin-1 (KHC)/Kinesin-5 (Eg5) constructs are shown in Table 1.1. The necklinker (both β9 and β10), coiled coil, and the elements β8 through α6 have been investigated [9, 10, 11, 12]. In examples disclosed herein, the coverstrand (β0 and loop 13 (L13)) was investigated. β9 of the necklinker was mutated to investigate the role of the cover neck bundle (CNB). A structural alignment was performed with PDB structures 1MKJ [13] for Kinesin-1 and 2WBE [14] for Kinesin-5 using the CE calculate two chains tool (http://cl.sdsc.edu/ce/ce_align.html). The two proteins aligned well, which can be seen in FIG. 2A. Regions of significant alignment were identified in the CS, NL, and L13. Places of diversion include extended loop2 (L2) and loop4 (L5) and shortened β4, β6, and β7. L5 has been the subject of investigation [15, 11] and is the target of the anticancer drug monastrol [16].

The mutations made to the k401 [6, 19] construct are shown in FIG. 2A. The sequences of Drosophila melanogaster kinesin heavy chain (KHC), Homo sapiens Kinesin-5 (Eg5), and the resulting chimeras are shown in Tables 1-2, 1-3, and 1-4 for the coverstrand, necklinker (β9 only), and L13, respectively. Notable amino acids differences between Eg5 and Kinesin-1 are the valine to proline in the necklinker and the asparagine to arginine in L13. The asparagine in L13 is highly conserved among a wide range of organisms for Kinesin-1.

### Kinetic Model Fits

A number of kinetic mechanisms were considered for fitting including the Boltzmann (equation 1.1), Fisher two state (equation 1.2), and Three State models (equation 1.3). The force-velocity data was fit by the three-state model described in [18], equation 1.3. The Boltzmann model

$$v ( F ) = \frac{{v}_{max} ( 1 + A )}{1 + A exp ( \frac{F δ}{{k}_{B} T} )} \qquad ( 1.1 )$$

has been used to model RNA polymerase [1] and kinesin [6] and uses the parameters vmax, A, and δ in the fit. In this model, A is the ratio of time of the mechanical component of the cycle to the biochemical component τₘ/τ_{b} and δ is the distance to the transition state. This distance is not necessarily the step size of the protein such as in the case of kinesin.

$$v ( F ) = d ( {u}_{0} {u}_{1} - {ω}_{0} {ω}_{1} ) / σ \\ σ = {u}_{0} + {u}_{1} + {ω}_{0} + {ω}_{1} \\ {u}_{0}^{0} = {k}_{0}^{0} [ ATP ] \\ u ( F ) = {u}_{j}^{0} exp ( - {θ}_{j}^{+} Fd / {k}_{B} T ) \\ {ω}_{0}^{0} = \frac{{k}_{0}^{j} [ ATP ]}{{( 1 + [ ATP ] / {c}_{0} )}^{1 / 2}} \\ ω ( F ) = {ω}_{j}^{0} exp ( + {θ}_{j}^{-} Fd / {k}_{B} T ) \\ {d}_{j} ( {θ}_{j}^{+} + {θ}_{j}^{-} ) d \qquad ( 1.2 )$$

The Fisher two state model [20, 21] splits the kinesin cycle into two states with forward and backward rates, which results in the cycle being split into four segments each with an associated rate. In this model, the u terms are forward reaction rates and the ω terms are the reverse rates. The θ terms are the characteristic fractions of the cycle that are occupied by each segment. The sum of all four θ values must equal one. In this model, each of the four rates are capable of being force dependent. The last model considered for fitting was the three state model used in [18].

$$v ( F ) = \frac{d {k}_{1} [ ATP ] {k}_{2} {k}_{3}}{{k}_{1} [ ATP ] ( {k}_{2} + {k}_{3} ) + {k}_{3} ( {k}_{2} + {k}_{- 1} )} \\ {k}_{2} = {k}_{2}^{0} exp ( - {F}_{2}^{δ} / {k}_{B} T ) \qquad ( 1.3 )$$

The rates k₁, k₋₁, k₂, and k₃ are for ATP binding, ATP dissociation, the mechanical step, and ATP hydrolysis, respectively. k₂⁰ is the unloaded rate for the mechanical step and δ₂ is the characteristic distance to the transition state, as in the Boltzmann model.

Each of these models was used to fit the data to determine which would provide the greatest amount of information with simplest form. It was found that the Boltzmann model and the three state model fit the data with nearly identical curves. It was found that the rates of the biochemical (ATP hydrolysis) and the mechanical step could be extracted from the A value that was fitted by the Boltzmann model. The time for the biochemical step could be extracted by

$${τ}_{b} = \frac{8.2}{{v}_{max} ( 1 + A )} \qquad ( 1.4 )$$

the reciprocal of which is the rate of the biochemical step. The length of the mechanical step was found with:

τₘ=Aτ_{b} (1.5)

As with the biochemical step, the reciprocal of this time is the rate of the mechanical step. When these rates were compared with those that were obtained by the three state model it was observed that the rates agreed very well. The additional information that the three state model provided was the rates of ATP binding and dissociation. These rates were globally fit for all of the chimeras and the wild type motors. For this particular analysis, it was assumed that the mutations do not affect the ATP binding domain of the proteins. This should be a reasonable assumption for this particular analysis because the ATP binding domain is located far from any of the mutated regions. The global fit for the ATP binding and dissociation constants resulted in values that were consistent with those that had been previously published [22]. There were challenges in using the Fisher two state model for fitting the data due to the large number of parameters to be fitted (nine parameters), and the requirement for all of the A values to sum to one. For these reasons, the three state model was selected for fitting the data in this investigation.

### Results

### Stall Force Measurements

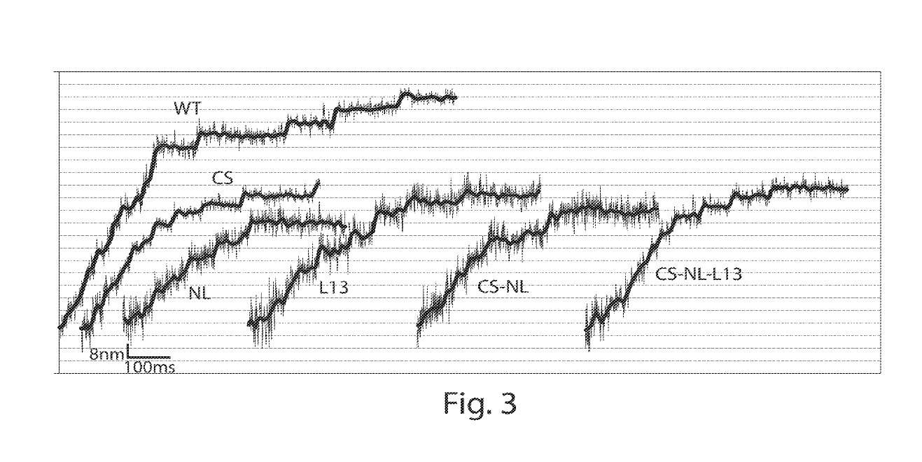

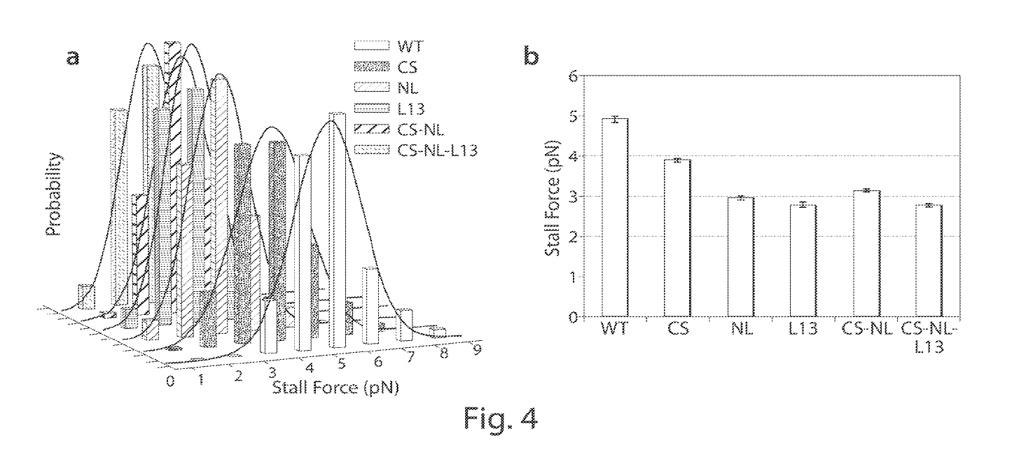

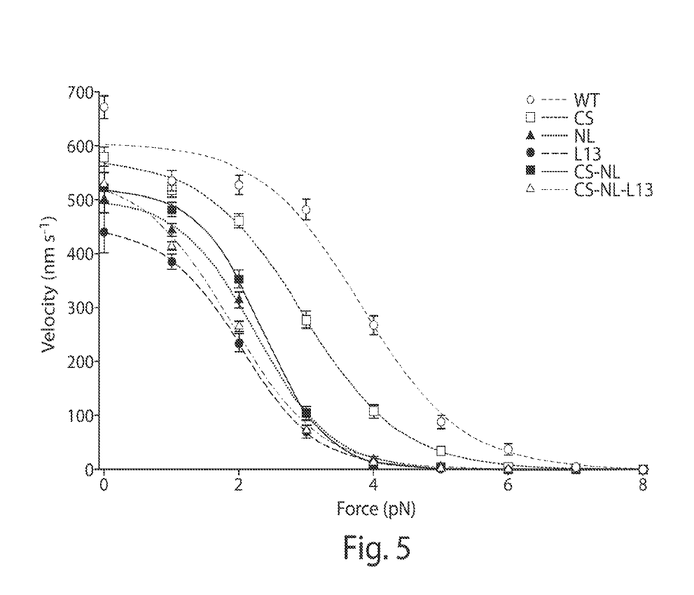

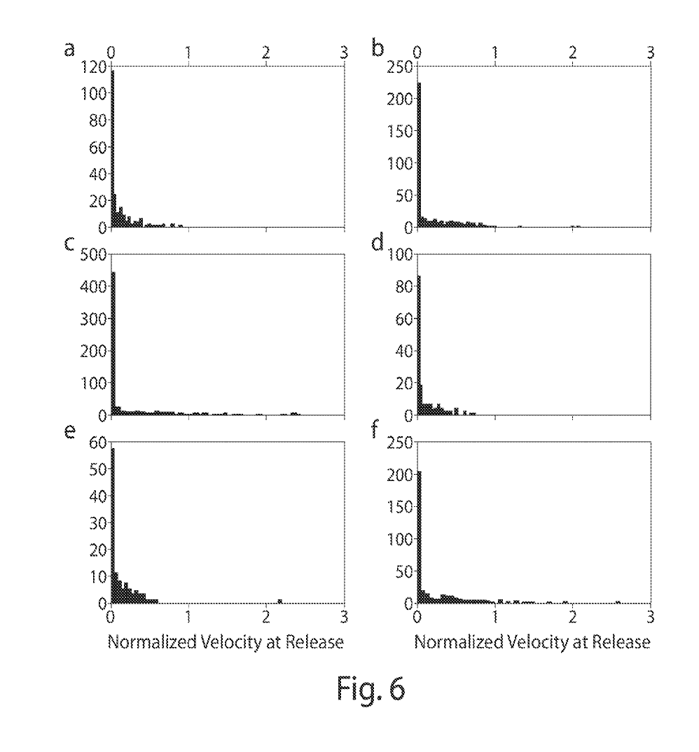

Representative runs for the various chimeric kinesin constructs are shown in FIG. 3. It was found that all chimeras take well defined steps of 8 nm, and each of the motors come to a well defined stall. The stall forces obtained by optical trapping experiments are shown in FIG. 4. Each of the runs was broken into 15 ms segments in which the average force was measured as well as average velocity, which was calculated by fitting a line to the position as a function of time data. These data were used to generate the force-velocity relationships seen in FIG. 5. The determination of the force-velocity information from stall force data was considered in this analysis to be a lower bound [23, 8] because velocities are calculated by assuming, for this analysis, that for each run the velocities above the force where the motor stalls is assumed to be zero. The force-velocity data was globally fit using the three state model described in the previous section. The parameters returned by these fits appear in Table 1.5. The stall forces were the mean values plus or minus the standard error of the mean. To determine whether the chimeric motors indeed stalled or rather released before a true stall was encountered, the velocity distribution at microtubule release was calculated. The histograms of velocities at stall for each of the kinesin constructs used in this study is shown in FIG. 6. The velocities were normalized to the unloaded velocity of each motor for comparison.

### Unloaded Measurements

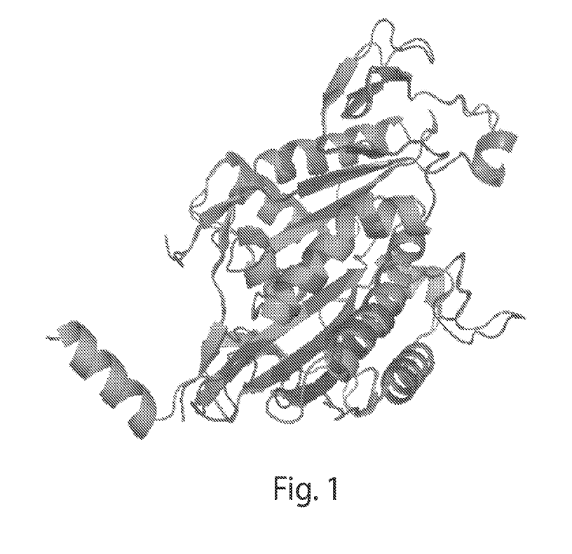

This study aimed to investigate the CNB model of force generation, and to develop a kinesin motor that was able to achieve very high force generating capability by combining the ability of Kinesin-1 to stall, with the ability of Eg5 to generate high forces without stalling. This aim of engineering a more powerful kinesin motor came with the assumption that the individual parts of proteins would be interchangeable, and that that the motor's characteristics are a sum of the contributions from each of the individual components. To test the above hypothesis, chimeric Kinesin-1/Eg5 constructs were developed that employed all permutations of the Eg5 CNB as well as the Eg5 L13. L13 was also chosen for investigation. This loop sits directly below the CNB, and may obstruct CNB mediated docking of the necklinker to the motorhead [3]. Further, L13 forms specific contacts with β9. Additionally, mutation of residues in L13 caused severe defects in motility [26]. The highly conserved glycine residues in L13 when mutated to alanine (G291A/G292A) reduced motility. This reduction may be due to a reduction in flexibility in L13 [3]. The location of these mutations is shown in FIGS. 1 and 2.

A set of seven chimeras were designed, the plasmids generated, expressed in E. coli and purified. Due to constraints on time, the most important of these were characterized using a kinesin motility assay based on Optical Trapping. These constructs included CS, NL, L13, CS-NL, and CS-NL-L13 all of which are useful for studying the function of kinesin proteins. The naming of these constructs is such that the signifiers in the name designate which structural elements the chimera possess from Eg5. The chimeric sequences for each of the individual components are found in Tables 1-2, 1-3, and 1-4 for the coverstrand, (NL), and L13, respectively.

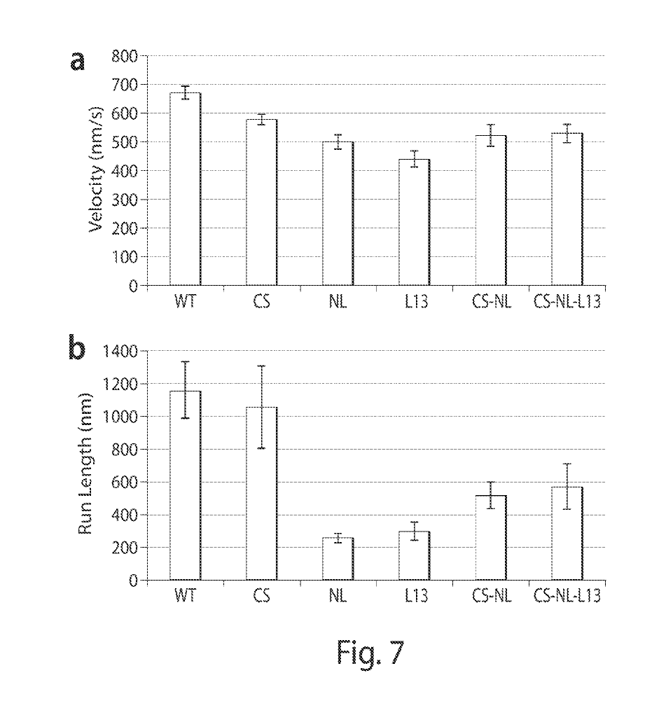

None of the chimeric proteins was able to withstand forces as high as the wildtype motor, and were below the dissociation forces of the dimeric Eg5 constructs of [18, 11]. The stall forces generated by these motors are shown in FIG. 4 and Table 1.5. Each of the chimeric motors also had defects in the unloaded velocity and in unloaded run length (with the exclusion of the CS chimera in terms of unloaded run length). The unloaded characteristics of the motors is shown in FIG. 7 and Table 1.6. The motors all retained the ability to take 8 nm steps and reach full stall, as shown in FIGS. 3 and 6.

### Coverstrand

The results disclosed herein indicate that the coverstrand has the ability to affect the velocity of the motor in addition to force generation. Molecular dynamics simulations [3] suggest that CNB has a conformational bias to move towards the motorhead and that in crystal structures that are in the ADP state and thus do not have a formed CNB, when the missing necklinker and coiled coil helix are added, the same conformational bias of the CNB towards the motor head was observed. An autonomous behavior was seen here.

In this example, when the Eg5 coverstrand was used on an otherwise unmodified motor, the rate of the mechanical step was slightly reduced, but remained the same order of magnitude. This may indicate that as long as a coverstrand is present to form the CNB, it will fold forward to generate force. While the coverstrand may appear to be more general than some of the other parts of the motor, there is evidence that a matched CNB operates more efficiently. The motors where the coverstrand was matched to the correct β9 (WT compared to CS and CS-NL compared to NL) had higher stall forces and longer runs than motors where the coverstrand did not match β9. The unloaded velocity was not substantially affected by the matching of the coverstrand to β9. This result may relate to the fact that the unloaded velocity of the motor is highly dependent upon the catalytic rate of the motor (usually referred to as k_{cat}, called k₃ in the model used here for fitting the force-velocity data), and this rate did not differ significantly between the motors.

### Interaction of β9 and L13

The asparagine in the Kinesin-1 L13 interacts with the valine of the necklinker. These two residues are greatly different in Eg5. The major mutation in the necklinker between Kinesin-1 and Eg5 is the substitution of proline for valine. In the case of L13, the major mutation is arginine for asparagine. The interruption of this contact may relate to the observation that the NL (where the β9 comes from Eg5) and L13 (where L13 comes from Eg5) have the significantly reduced performance in certain contexts.

The reductions in force generating capabilities of the chimeras studied here may not be associated with defects in the latching action of the asparagine latch of kinesin. In the case of the Eg5 necklinker, a proline is present, which is known as a beta sheet breaker. This proline would appear to limit the size of the CNB, and thus potentially its force generation capability.

### L13 as a Stabilizer

L13 may act to stabilize the powerstroke when the CNB matches the L13. However when the CNB does not match the L13, as in the case of when the Eg5 L13 was mutated into Kinesin-1 or when the wildtype Kinesin-1 L13 was used with the Eg5 CNB, the L13 may act to destabilize the folding of the CNB toward the motorhead. This may relate to differences in contacts between β9 and L13. In the case of the L13 chimera, the arginine residue in place of the asparagine residue may attenuate force generation. The arginine is larger than the asparagine and may interfere with the CNB's fold toward the motorhead and the necklinker's docking to the motorhead.

The Eg5 L13 does not appear to significantly affect either unloaded velocity or run length when used with the Eg5 CNB, but it reduced both the unloaded velocity and run length when used with the Kinesin-1 CNB. The characteristic distance for the mechanical step, δ₂ is lower for the chimera that contains the mutated coverstrand, β9, and L13 (CS-NL-L13) than the construct containing the mutated coverstrand and β9 (CS-NL), which may suggest a decrease in force sensitivity on the mechanical rate. In the three state model, as with the Boltzmann model, the characteristic distance is may not be the full size of the step that the motor takes.

Structural Relationship with Stall Force

The characteristic distance may be a measure of force sensitivity, as it is used in the exponential term of the mechanical rate, as seen in equation 1.3. Upon inspection of the unloaded mechanical rate, it was observed that this rate is an order of magnitude faster for the CS-NL chimera than the CS-NL-L13 chimera. The unloaded mechanical rate, k₂⁰ for the chimeras with a matched CNB were the fastest, which may be due to fast formation and folding forward of the CNB, however in the case of the CS-NL chimera, the sensitivity to force is high, and this may be because the Eg5 L13 is not present to stabilize the CNB when it folds forward.

In the case of CS-NL-L13, the Eg5 L13 may cause CNB folding to be slower, but in the end stabilizes the CNB when it folds forward, thus producing a less force sensitive mechanical rate. The slower CNB folding (unloaded mechanical rate), may be the source of the lower stall force. The slower mechanical rates of these proteins may not significantly affect the velocity of the motors for most of kinesin's run, as the mechanical rate does not become limiting until the motor is nearly stalled. The rate of ATP hydrolysis, k₃ is typically much slower, and thus limits the velocity of the motors. The motors may have a mechanical rate of 24±4 s⁻¹ (average plus or minus the standard deviation) at the stall force. The force at which the mechanical rate becomes rate limiting (when it becomes slower than the rate of ATP hydrolysis) is 80±3% of the stall force. The consistency of the mechanical rate at stall and the fraction of the stall force where the mechanical rate is limiting among the motor constructs investigated, indicate that the mechanical rate and stall force are related.

Furthermore, when the data for the constructs where two glycines were mutated into the coverstrand and where the coverstrand was deleted [6] were analyzed in this way, it was found that they also had mechanical rates at stall on the order of tens per second and that the force at which the mechanical rate became limiting was above 80%. Similar results were also found with the dimeric Eg5 data of [18, 11]. These results are shown in Table 1.7.

An indication of a link between the mechanical rate and the stall force may come from kinesin's dissociation rate from microtubules. It has been observed that this dissociation rate is force dependent, and at forces that are close to these constructs' stall force, the dissociation rate is on the order of 1 s⁻¹ (K S Thorn, J A Ubersax, and R D Vale. Engineering the processive run length of the kinesin motor. The Journal of Cell Biology, 151(5):1093-100, November 2000). While this is about an order of magnitude smaller than the mechanical rate at stall, it may be that the slow mechanical rate at stall allows for a higher probability of dissociation from the microtubule before the completion of the full mechanochemical cycle. This link between the mechanical rate of the motor and the stall force provides a basis for engineering kinesin motors to have a prescribed stall force. For example, by making the CNB fold forward faster (or have less force sensitivity), the force at which the mechanical rate becomes limiting would be higher, thereby increasing the stall force.

### Further Observations

The generation of Kinesin-1/Eg5 chimeras using elements from kinesin's proposed force generation mechanism, the CNB (the coverstrand and β9) as well as a loop that is known to interact with β9, L13, have provided additional views into the force generation mechanism of kinesin. This model has been expanded to include effects of L13, which appears to have a stabilizing effect, and that this effect is likely due to contacts between β9 and L13, such as N 327 and T328 of β9 with L290, G291, G292 of L13 (human KHC numbering used) and V329 of β9 with N293 of L13.

Furthermore, changing the sequences of the components of the CNB and L13, results in a change in the rate of the mechanical step. This change in mechanical rate may relate to the observed differences in stall force. Mutation of structural elements of Kinesin-1 to that of those containing the sequences from Eg5 may attenuate kinesin force generation capabilities and performance, even when all of the elements associated with force generation are mutated to the Eg5 sequences. Comparing these results to the reported force generating capabilities of dimeric constructs of Eg5 show that while the CNB is important, and that a matched CNB works efficiently, there may be more to the mechanism of force generation than is explained by the CNB model. The force generation characteristics of Eg5 may be captured using a chimera that includes α6, the alpha helix directly preceding β9, from Eg5.

Methods are presented in Example 3.

### Figures for Example 1

FIG. 1 depicts Structural alignments of Kinesin-1 and Kinesin-5 (Eg5). Kinesin-1 is shown in red and Eg5 in gray. The Figure illustrates that the significant alignment between the structures, particularly in the coverstrand, necklinker, and L13. Major departures from alignment in the two proteins were observed at loops 2 and 5 as well as in the beta sheets at the front tip of the motor (β4, β6, and β7).

FIG. 2 depicts chimeric sequences used in this study. The fruit fly sequence of the wildtype protein, the human Eg5 sequence, and the resulting chimeric sequence for each of the locations of mutations (coverstrand, green; β9, blue; and L13, red) are shown. ATP is shown in the sphere representation. PDB 1MKJ was used to generate this Figure.

FIG. 3 depicts representative traces of runs from each of the constructs used herein. Wildtype is shown in cyan, CS in blue, NL in red, L13 in yellow, CS-NL in brown, and CS-NL-L13 in turquoise. In each case, the proteins took well defined 8 nm steps and had well defined stall plateaus. The scale bars represent 8 nm in vertical direction and 100 ms in the horizontal direction.

FIG. 4 depicts stall forces of each of the kinesin constructs used in this study. FIG. 4A shows histograms of the stall forces obtained from stationary trap experiments. As can be seen, each of these distributions can be fit with a normal distribution (curve) very well. FIG. 4b shows the average stall forces for each of the kinesin constructs. The error bars in FIG. 4b are plus/minus the standard error of the mean. The wildtype motor produced the most force with a stall force of approximately 5 pN, while all of the chimeric proteins produced less force. The numerical values of the stall force are shown in Table 1.5.

FIG. 5 shows the force-velocity behavior of the kinesin constructs used in this study.

The symbols are the data obtained by mathematical treatment of the stall force data, as described herein, except for the velocities at zero force, which were obtained via the unloaded velocity measurements. The error bars are standard error of the mean of the data in each force bin. The data was fit with the three state model, equation 1.3. The unloaded velocities and the data obtained from the stall force measurements were used in fitting the data.

FIG. 6 shows the distributions of velocity at stall from the stall force data. The distributions in FIG. 6 are as follows a) WT b) CS c) NL d) L13 e) CS-NL f) CS-NL-L13. These distributions were obtained by manually fitting a line to time-displacement data obtained from the stall force measurements to the last few moments before dissociation. If the slope of this line was negative (backwards motion), the velocity was assumed, for the analysis, to be zero. Each of the distributions was normalized to the unloaded velocity of the respective motor. As can be seen, these motors all had a sharp peak in dissociation velocity at very low speeds and the majority of dissociations occurred below the unloaded velocity.

FIG. 7 shows that the unloaded velocity was relatively low for all of the chimeras. All of the velocities were well above those found for wildtype Eg5 (around 100 nm s⁻¹). The run lengths of the NL and L13 constructs was approximately that of the construct with a deleted coverstrand [6]. The presence of the paired coverstrand (CS-NL) and (CS-NL-L13) recovers some run length, but only to a value about half of the wildtype motor. The numerical values for these bar plots are shown in Table 1.6.

### Tables for Example 1

TABLE 1.1
Studies using Kinesin-1/Kinesin-5 chimeras.
The construct name, the origin of each the structural element, a description of the
chimera, and the reference from which the chimer originated is provided. The present study
investigates the effects of the coverstrand and loop 13 (L13). Constructs used in this work are
designated, WT, CS, NL, L13, CS-NL, CS-NL-L13. K = Kinesin-1; E = Eg 5, Kinesin-5

| | Cover- | | | | | | | | Coiled | | | |
|---|---|---|---|---|---|---|---|---|---|---|---|---|
| | strand | | Core | | β9 | | β10 | | Coil | | | |
| Construct | K | E | K | E | K | E | K | E | K | E | Notes | Ref |
| K401 (WT) | X | | X | | X | | X | | X | | D. melanogaster | This study, |
| | | | | | | | | | | | KHC up to as401 | [6] |
| | | | | | | | | | | | (first hinge) | |
| Eg5 | | X | | X | | X | | X | | X | X. laevis Full | [17]/[18] |
| | | | | | | | | | | | length, tetrameric | |
| | | | | | | | | | | | Eg5/Up to aa513, | |
| | | | | | | | | | | | dimer | |
| CS | | X | X | | X | | X | | X | | K401 with Eg5 CS | This study |
| NL | X | | X | | | X | X | | X | | K401 with Eg5 β9 | This study |
| L13 | X | | X | L13 | X | | X | | X | | K401 with Eg5 L13 | This study |
| CS-NL | | X | X | | | X | X | | X | | K401 with Eg5 CS | This study |
| | | | | | | | | | | | and β9 | |
| CS-NL-L13 | | X | X | L13 | | X | X | | X | | K401 with Eg5 CS, | This study |
| | | | | | | | | | | | β9, and L13 | |
| K-E (necklinker) | X | | X | | | X | | X | | X | H. sapiens KHC | [9, 11] |
| | | | | | | | | | | | with Eg5 β9, β9, | |
| | | | | | | | | | | | coiled coil | |
| E-K (necklinker) | | X | | X | X | | X | | X | | Eg5 motorhead | [9] |
| | | | | | | | | | | | with H. sapiens | |
| | | | | | | | | | | | KHC β9, β9, coiled | |
| | | | | | | | | | | | coil to aa560 | |
| K-E (neck) | X | | X | | X | | X | | | X | D. melanogaster | [9] |
| | | | | | | | | | | | KHC with Eg5 | |
| | | | | | | | | | | | coiled coil | |
| E-K (neck) | | X | | X | | X | | X | X | | Eg5 with H. sapiens | [9] |
| | | | | | | | | | | | KHC coiled | |
| | | | | | | | | | | | coil | |
| K (5aa linker)- | X | | X | | T1KNT | X | | X | | X | H. sapiens KHC up | [9] |
| E(necklinker) | | | | | | | | | | | to middle β9 with | |
| | | | | | | | | | | | Egt remainder of | |
| | | | | | | | | | | | β9, β9, coiled coil | |
| | | | | | | | | | | | to aa513 | |
| K-E (β8α6- | X | | X | β8α6 | | X | | X | | X | H. sapiens KHC up | [10] |
| necklinker) | | | | | | | | | | | to β8, Eg5 β8 | |
| | | | | | | | | | | | onwards | |
| E-K (β8α6- | | X | β8α6 | X | | X | | X | | X | Eg5 up to β8, H. sapiens | [10] |
| necklinker) | | | | | | | | | | | KHC β8 | |
| | | | | | | | | | | | onwards | |
| DK4mer | X | | X | | X | | X | | | X | D. melanogaster | [12] |
| | | | | | | | | | | | KHC with Eg5 full | |
| | | | | | | | | | | | length coiled coil, | |
| | | | | | | | | | | | tetrameric | |

TABLE 1.2
Sequence comparison for the coverstrand.
An alignment was made between human Kinesin-1
(KHC), fruit fly KHC, and human Kinesin-5 (Eg5).
The isoleucine at the end of the coverstrand
is conserved in the listed proteins.

| | | | | | | | | | | | | | | | | | * | * | * |
|---|---|---|---|---|---|---|---|---|---|---|---|---|---|---|---|---|---|---|---|
| H. sapiens KHC | — | — | — | — | — | — | — | — | — | — | M | A | D | L | A | E | C | N | I⁹ |
| (SEQ ID NO: 8) | | | | | | | | | | | | | | | | | | | |
| D. melanogaster | — | — | — | — | — | — | M | S | A | E | R | E | I | P | A | E | D | S | I¹³ |
| KHC | | | | | | | | | | | | | | | | | | | |
| (SEQ ID NO: 9) | | | | | | | | | | | | | | | | | | | |
| H. sapiens Eg5 | M | A | S | Q | P | N | S | S | A | K | K | K | E | E | K | G | K | N | I¹⁹ |
| (SEQ ID NO: 10) | | | | | | | | | | | | | | | | | | | |

TABLE 1.3
Sequence comparison for the β9
segment of the necklinker.
The same sequence alignment was used as in Table 2.
β9 corresponds to the segment between the far left
isoleucine or valine to asparagine 332 for human
KHC (340 for fruit fly KHC and 365 for human Eg5).
Also of note is the proline residue in Eg5 in place
of the conserved valine in KHC. Proline is known
to act as a beta sheet breaker, thus
limiting the size of β9

| | | | | | | | | | | | | |
|---|---|---|---|---|---|---|---|---|---|---|---|---|
| H. sapiens KHC | I³²⁵ | K | N | T | V | C | V | N³³² | V | E | L | T |
| (SEQ ID NO: 11) | | | | | | | | | | | | |
| D. melanogaster | V³³³ | K | N | V | V | C | V | N³⁴⁰ | E | E | L | T |
| KHC | | | | | | | | | | | | |
| (SEQ ID NO: 12) | | | | | | | | | | | | |
| H. sapiens Eg5 | I³⁵⁹ | L | N | K | P | E | V | N³⁶⁶ | Q | K | — | — |
| (SEQ ID NO: 13) | | | | | | | | | | | | |

TABLE 1.4
Sequence comparison for loop 13 (L13).
The same sequence alignment was used as in Table 2.
L13 has an arginine in Eg5 in place of the
conserved asparagine in the KHC sequences. As can
be seen, much of L13 is highly conserved.

| | | | | | | |
|---|---|---|---|---|---|---|
| H. sapiens KHC | L²⁹⁰ | G | G | N | C | R²⁹⁵ |
| (SEQ ID NO: 14) | | | | | | |
| D. melanogaster | L²⁹⁸ | G | G | N | A | R³⁰³ |
| KHC | | | | | | |
| (SEQ ID NO: 15) | | | | | | |
| H. sapiens Eg5 | L³²⁴ | G | G | R | T | R³²⁹ |
| (SEQ ID NO: 16) | | | | | | |

TABLE 1.5
Stall force and fitted parameters for force-velocity data for each of the
constructs used in this study.
The three state model (equation 1.3 was used to fit the force-velocity data
shown in FIG. 5 to obtain the values shown here. The stall force is
mean plus/minus standard error of the mean. The ATP binding and
dissociation rates (k₁ and k₋₁ were globally fit to all of the motors).

| | | Three State Model | | | | | | |
|---|---|---|---|---|---|---|---|---|
| | Stall Force | k₁ | k₋₁ | k₂⁰ | kₛ | δ₂ | K_{M}⁰ | νₘₐₓ |
| | (pN) | (μM⁻¹ · s⁻¹) | (s⁻¹) | (s⁻¹) | (s⁻¹) | (nm) | (μM) | (nm · s⁻¹) |
| WT | 4.92 ± 0.08 | 1.25 | 619.77 | 18400 | 78.65 | 5.51 | 63.08 | 644.95 |
| CS | 3.89 ± 0.05 | 1.25 | 619.77 | 6030 | 74.37 | 5.65 | 59.65 | 609.84 |
| NL | 2.95 ± 0.05 | 1.25 | 619.77 | 5150 | 64.43 | 7.35 | 51.68 | 528.32 |
| L13 | 2.79 ± 0.06 | 1.25 | 619.77 | 3000 | 57.41 | 7.27 | 46.04 | 470.75 |
| CS-NL | 3.15 ± 0.04 | 1.25 | 619.77 | 13400 | 66.67 | 8.69 | 53.47 | 546.66 |
| CS-NL-L13 | 2.78 ± 0.03 | 1.25 | 619.77 | 1690 | 70.90 | 6.22 | 56.87 | 581.39 |

TABLE 1.6
Unloaded velocities and run lengths for each of the constructs
used in this study. The data listed is are the average plus or
minus the standard error of the mean. This data
is visualized in FIG. 7.

| | v⁰(nm · δ⁻¹) | Run Length (nm) |
|---|---|---|
| WT | 671.27 ± 21.15 | 1163.32 ± 172.08 |
| CS | 579.24 ± 17.70 | 1056.80 ± 252.70 |
| NL | 500.67 ± 25.84 | 259.67 ± 27.12 |
| L13 | 439.70 ± 37.85 | 300.94 ± 56.12 |
| CS-NL | 521.98 ± 28.17 | 518.44 ± 79.19 |
| Cs-NL-L13 | 528.82 ± 32.86 | 573.59 ± 137.67 |

TABLE 1.7
Rates for the mechanical step of kinesin's mechanochemical cycle.
The stall force, unloaded mechanical rate and δ2 come from Table 3.5. The mechanical
rate at stall (k₂^{stall} were calculated by using the stall force, k₂⁰, and δ2 for
each motor. The force at which the mechanical step becomes limiting, F_{mechlimiting}
was calculated by rearranging the second equation in Equation 1.3 to solve for force
and plugging in k₃, the catalytic rate, in place of k₂.

| | Fstall (pN) | k₂⁰(s⁻¹) | δ₂ | kₛ^{stall}(s⁻¹) | F_{mechlimiting (pN)} | F_{mechlimiting}/Fₛₜₐₗₗ (%) |
|---|---|---|---|---|---|---|
| WT | 4.92 | 18400 | 5.51 | 25.40 | 4.08 | 82.4 |
| CS | 3.89 | 6030 | 5.65 | 28.86 | 3.20 | 82.3 |
| NL | 2.95 | 5150 | 7.35 | 26.47 | 2.45 | 83.1 |
| L13 | 2.79 | 3000 | 7.27 | 21.70 | 2.24 | 80.2 |
| CS-NL | 3.15 | 13400 | 8.69 | 17.23 | 2.51 | 79.7 |
| CS-NL-L13 | 2.789 | 1690 | 6.22 | 25.21 | 2.10 | 75.4 |
| 2G [6] | 3.02 | 4830 | 7.15 | 39.97 | 2.47 | 81.8 |
| DEL [6] | 1.37 | 1710 | 11.28 | 25.39 | 1.22 | 88.8 |
| Eg5 [18] | 4.6 [11] | 86 | 1.9 | 10.28 | 4.01 | 87.2 |

```
Sequences of Chimeric Proteins
(SEQ ID NO: 1) WT (Drosophila melanogaster KHC full sequence):
                10 20 30 40 50 60
MSAEREIPAE DSIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
NARTTIVICC SPASFNESET KSTLDFGRRA KTVKNVVCVN EELTAEEWKR RYEKEKEKNA
              370 380 390 400 410 420
RLKGKVEKLE IELARWRAGE TVKAEEQINM EDLMEASTPN LEVEAAQTAA AEAALAAQRT
              430 440 450 460 470 480
ALANMSASVA VNEQARLATE CERLYQQLDD KDEEINQQSQ YAEQLKEQVM EQEELIANAR
              490 500 510 520 530 540
REYETLQSEM ARIQQENESA KEEVKEVLQA LEELAVNYDQ KSQEIDNKNK DIDALNEELQ
              550 560 570 580 590 600
QKQSVFNAAS TELQQLKDMS SHQKKRITEM LTNLLRDLGE VGQAIAPGES SIDLKMSALA
              610 620 630 640 650 660
GTDASKVEED FTMARLFISK MKTEAKNIAQ RCSNMETQQA DSNKKISEYE KDLGEYRLLI
              670 680 690 700 710 720
SQHEARMKSL QESMREAENK KRTLEEQIDS LREECAKLKA AEHVSAVNAE EKQRAEELRS
              730 740 750 760 770 780
MFDSQMDELR EAHTRQVSEL RDEIAAKQHE MDEMKDVHQK LLLAHQQMTA DYEKVRQEDA
              790 800 810 820 830 840
EKSSELQNII LTNERREQAR KDLKGLEDTV AKELQTLHNL RKLFVQDLQQ RIRKNVVNEE
              850 860 870 880 890 900
SEEDGGSLAQ KQKISFLENN LDQLTKVHKQ LVRDNADLRC ELPKLEKRLR CTMERVKALE
              910 920 930 940 950 960
TALKEAKEGA MRDRKRYQYE VDRIKEAVRQ KHLGRRGPQA QIAKPIRSGQ GAIAIRGGGA
              970
VGGPSPLAQV NPVNS
(SEQ ID NO: 2) WT (K401-Bio-His6)
                10 20 30 40 50 60
MSAEREIPAE DSIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
NARTTIVICC SPASFNESET KSTLDFGRRA KTVKNVVCVN EELTAEEWKR RYEKEKEKNA
              370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 3) CS
                10 20 30 40 50 60
MSAKKKEEKG KNIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
NARTTIVICC SPASFNESET KSTLDFGRRA KTVKNVVCVN EELTAEEWKR RYEKEKEKNA
              370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 4) NL
                10 20 30 40 50 60
MSAEREIPAE DSIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
NARTTIVICC SPASFNESET KSTLDFGRRA KTILNKPEVN EELTAEEWKR RYEKEKEKNA
              370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 5) L13
                10 20 30 40 50 60
MSAEREIPAE DSIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
RTRTTIVICC SPASFNESET KSTLDFGRRA KTVKNVVCVN EELTAEEWKR RYEKEKEKNA
                370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 6) CS-NL
                10 20 30 40 50 60
MSAKKKEEKG KNIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
NARTTIVICC SPASFNESET KSTLDFGRRA KTILNKPEVN EELTAEEWKR RYEKEKEKNA
              370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 7) CS-NL-L13
                10 20 30 40 50 60
MSAKKKEEKG KNIKVVCRFR PLNDSEEKAG SKFVVKFPNN VEENCISIAG KVYLFDKVFK
                70 80 90 100 110 120
PNASQEKVYN EAAKSIVTDV LAGYNGTIFA YGQTSSGKTH TMEGVIGDSV KQGIIPRIVN
              130 140 150 160 170 180
DIFNHIYAME VNLEFHIKVS YYEIYMDKIR DLLDVSKVNL SVHEDKNRVP YVKGATERFV
              190 200 210 220 230 240
SSPEDVFEVI EEGKSNRHIA VTNMNEHSSR SHSVFLINVK QENLENQKKL SGKLYLVDLA
              250 260 270 280 290 300
GSEKVSKTGA EGTVLDEAKN INKSLSALGN VISALADGNK THIPYRDSKL TRILQESLGG
              310 320 330 340 350 360
RTRTTIVICC SPASFNESET KSTLDFGRRA KTILNKPEVN EELTAEEWKR RYEKEKEKNA
              370 380 390 400 410 420
RLKGKVEKLE IELARWRAGE TVKAEEQINM EDLMEASTPN LRKAMEAPAA AEISGHIVRS
              430 440 450 460 470 480
PMVGTFYRTP SPDAKAFIEV GQKVNVGDTL CIVEAMKMMN QIEADKSGTV KAILVESGQP
              490 500
VEFDEPLVVI ELSETSGHHH HHH
(SEQ ID NO: 20 - Kinesin-5 (Eg5))
>gi|13699824|ref|NP_004514.2| kinesin-like protein KIF11 [Homo sapiens]
MASQPNSSAKKKEEKGKNIQVVVRCRPFNLAERKASAHSIVECDPVRKEVSVRTGGLADKSSRKTYTFDM
VFGASTKQIDVYRSVVCPILDEVIMGYNCTIFAYGQTGTGKTFTMEGERSPNEEYTWEEDPLAGIIPRTL
HQIFEKLTDNGTEFSVKVSLLEIYNEELFDLLNPSSDVSERLQMFDDPRNKRGVIIKGLEEITVHNKDEV
YQILEKGAAKRTTAATLMNAYSSRSHSVFSVTIHMKETTIDGEELVKIGKLNLVDLAGSENIGRSGAVDK
RAREAGNINQSLLTLGRVITALVERTPHVPYRESKLTRILQDSLGGRTRTSIIATISPASLNLEETLSTL
EYAHRAKNILNKPEVNQKLTKKALIKEYTEEIERLKRDLAAAREKNGVYISEENFRVMSGKLTVQEEQIV
ELIEKIGAVEEELNRVTELFMDNKNELDQCKSDLQNKTQELETTQKHLQETKLQLVKEEYITSALESTEE
KLHDAASKLLNTVEETTKDVSGLHSKLDRKKAVDQHNAEAQDIFGKNLNSLFNNMEELIKDGSSKQKAML
EVHKTLFGNLLSSSVSALDTITTVALGSLTSIPENVSTHVSQIFNMILKEQSLAAESKTVLQELINVLKT
DLLSSLEMILSPTVVSILKINSQLKHIFKTSLTVADKIEDQKKELDGFLSILCNNLHELQENTICSLVES
QKQCGNLTEDLKTIKQTHSQELCKLMNLWTERFCALEEKCENIQKPLSSVQENIQQKSKDIVNKMTFHSQ
KFCADSDGFSQELRNFNQEGTKLVEESVKHSDKLNGNLEKISQETEQRCESLNTRTVYFSEQWVSSLNER
EQELHNLLEVVSQCCEASSSDITEKSDGRKAAHEKQHNIFLDQMTIDEDKLIAQNLELNETIKIGLTKLN
CFLEQDLKLDIPTGTTPQRKSYLYPSTLVRTEPREHLLDQLKRKQPELLMMLNCSENNKEETIPDVDVEE
AVLGQYTEEPLSQEPSVDAGVDCSSIGGVPFFQHKKSHGKDKENRGINTLERSKVEETTEHLVTKSRLPL
RAQINL
```

### Example 2

### Antibody Inhibition of Kinesin Activity

The CNB mechanism of force generation was used as a target for designing an antibody that inhibits kinesin motility. Antibodies were generated using synthesized peptides corresponding to the coverstrand of Kinesin-1 of D. melanogaster. Two peptide sequences were used, see Table 2.1. Version 1 includes less of the sequence of the coverstrand and allows for less specific targeting of the coverstrand. The second version covers the full coverstrand, and was designed so that the antibody would be more specific for the coverstrand. A cysteine residue was added to the C-terminus of each peptide to attach the peptides to a substrate for immunization. Four rabbits were immunized with both version of the peptides with four injections of mixtures of both versions of the peptide in the schedule shown in Table 2.2.

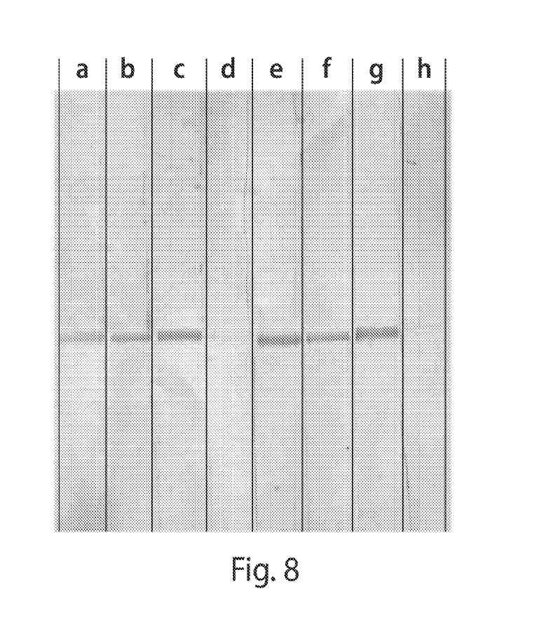

The version 2 peptide was used for affinity purification of the antisera. The rabbits were then bled to obtain antisera that was used to determine which bleeds should be used for antibody purification. Bleeds 1 and 3 or 2 and 4 were identified as being satisfactory for purification determination. A western blot was run using bleeds 2 and 4 from each of the four rabbits, which is shown in FIG. 8. From this western blot, it was determined that bleed 4 from rabbits 4078 and 4080 would be used for purification. The purified sera was combined and used for all of the experiments described herein.

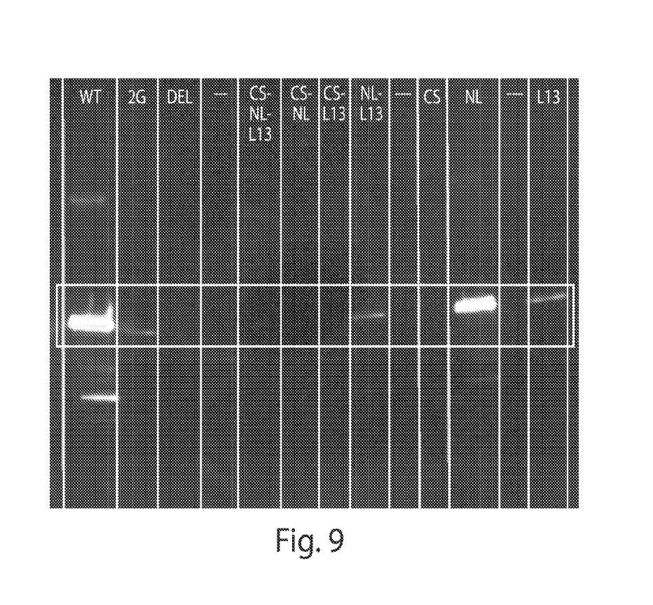

The western blot showing that the antibody was specifically targeted to coverstrands with the WT sequence is shown in FIG. 9. The difference in fluorescence of the bands is due to unequal loading of kinesin into the gel used for separating the protein. Here it is observed that kinesin constructs in which a Kinesin-1 coverstrand exists are targeted by the antibody, and thus become fluorescent. The 2G construct, where two glycine residues were mutated into the coverstrand was also targeted. This was expected as the majority of the residues were the same as the wild type protein, and that the glycines should make the coverstrand more flexible, and thus perhaps make the rest of the residues more easily identified by the antibody.

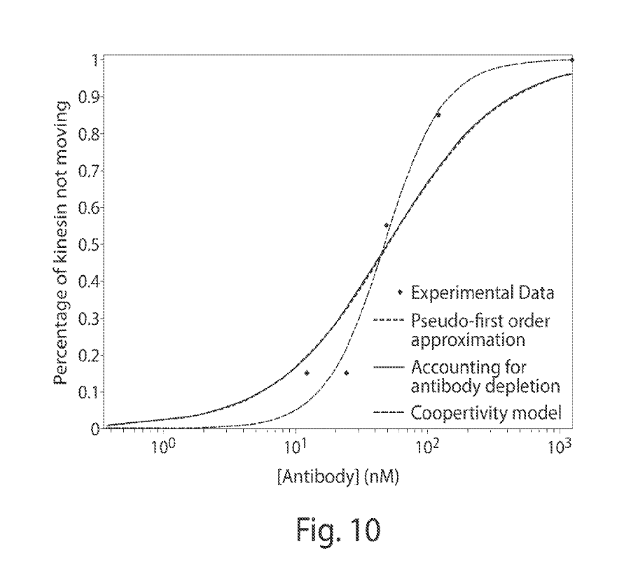

FIG. 10 shows the concentration dependence on the inhibition of kinesin motion. In this experiment, the kinesin were incubated with the concentration of antibody to be tested for fifteen minutes on ice before use. The concentrations of kinesin used in each of these experiments was slightly above the single molecule limit, where either all or nearly all beads were motile, but not such high concentrations that the beads would simply stick to the microtubule and not move in the absence of antibody. Both the wild type Kinesin-1 and CS chimera were used to determine the efficacy of the antibody to inhibit kinesin motion. Only beads that became tethered to the microtubule after being placed next to the microtubule for a few seconds were considered for analysis. Beads that tethered and at some point began running were not considered as being inhibited. Twenty beads were tested at each concentration of antibody. The CS chimera was tested with no antibody and with the highest concentration of antibody that was tested for WT Kinesin-1. The CS construct displayed similar motility as in the unloaded assay described in Example 3, regardless of the concentration of antibody that was present. Thus, no antibody related tethering was noticed with the CS construct, which confirmed that the antibody's effect on Kinesin-1's motility is specific to the antibody binding to the coverstrand and not a nonspecific interaction such as interaction with the microtubules or glass slide.

### Kinesin-5 Antibodies

The following peptides were used to generate a rabbit polyclonal antibody against the human Kinesin-5 coverstrand (Eg5): MSAKKKEEKGKNI (SEQ ID NO: 17) and MASQPNSSAKKKEEKGKNI (SEQ ID NO: 23). The first sequence (SEQ ID NO: 17) corresponds to a chimeric kinesin created with a Eg5 coverstrand and kinesin-1. The full length Eg5 coverstrand was truncated to be the same length as Kinesin-1 (from D. melanogaster) coverstrand. The second sequence (SEQ ID NO: 23) corresponds to the full length Eg5 coverstrand. A cysteine residue was added to the C-terminus of each peptide to attach the peptides to a substrate for immunization. Four rabbits were immunized with both versions of the peptides using four injections of mixtures of both versions of the peptide.

A mouse monoclonal antibody was prepared using standard techniques with the following peptide: EKGKNI (SEQ ID NO: 24).

### Observations

The western blot shows that the antibody can target the kinesin constructs that have the wild type kinesin I coverstrand. The concentration dependent inhibition of kinesin motility was fit using

$$y = \frac{[ AB ]}{[ AB ] + {K}_{D}} \qquad ( 2.1 )$$

where y is the fraction of beads that are not motile, and the pseudo-first order approximation is used. Under the pseudo-first order approximation, the concentration of ligand (antibody in this case) is assumed to be in a great enough excess that it can be considered to be constant. The data were also fit with the same relation, but accounting for antibody depletion with the following formula

$$y = \frac{\begin{matrix}( {K}_{D} + [ AB ] + [ kine sin ] ) - \\ \sqrt{{( {K}_{D} + [ AB ] + [ kine sin ] )}^{2} - 4 [ kine sin e ] [ AB ]}\end{matrix}}{2 [ kine sin ]} \qquad ( 2.2 )$$

Finally the data were also fit using a relation that allows for cooperativity.

$$y = \frac{{[ AB ]}^{n}}{{[ AB ]}^{n} + {K}_{D}} \qquad ( 2.3 )$$

The parameters determined by these fits are shown in Table 2.4. As can be seen the model that allows for cooperativity fits the data the best, and that the depletion of antibody is not significant during the experiment, and thus the pseudo-first order approximation is applicable, as accounting for antibody depletion does not change the fitted equilibrium dissociation constant, KD. Interestingly, the cooperativity model suggests that two antibodies can bind to kinesin. This result makes sense as there are two motor heads per kinesin molecule, and thus two coverstrands per molecule that can bind antibody. The model that includes cooperativity fits the data most reliably, which suggests that the antibody does not bind very tightly to kinesin, since it has a micromolar equilibrium dissociation constant.

The antibody binds to the coverstrand and inhibits the ability for kinesin to move. This appears to be due to the antibody binding to the coverstrand, which then obstructs the formation of the cover neck bundle, thus inhibiting the force generation mechanism of kinesin. It is also known from experiments with the CS chimera, which is identical to the Kinesin-1 construct that was tested except for the coverstrand, that the antibody's effect on motility is specific to the CNB formation. Further work must be done to determine the exact mechanism of force generation inhibition in kinesin. The discussion of these further studies can be found in chapter 5. It is expected that the antibody binds to the coverstrand and thus sterically inhibits CNB formation. It is still unknown however, where in the kinesin cycle that the antibody binds to the coverstrand, and how this affects the ATPase cycle of the motor. It would make sense that the antibody would interact with the coverstrand in either the empty or ADP state, as these states correspond to states where the coverstrand is not interacting with β9. Since the bead appears to tether as soon as it interacts with the micro-tubule, and that the kinesin was allowed to incubate with the antibodies for some time before the experiment was started, it is believed that the antibody first binds to the kinesin in the empty state before interaction with the microtubule. This is because the empty state is a strong microtubule binder and the ADP state is not.

### Example 2

### Figures

FIG. 8: Western blot used for determination of bleeds to use for purification. Two bleeds from each of the four rabbits were used, wild type Kinesin-1 from D. melanogaster was used as the target protein. The lanes are as follows a) bleed 2 from rabbit 4078 b) bleed 2 from 4079 c) bleed 2 from 4080 d) bleed 2 from 4081, e) bleed 4 from 4078 f) bleed 4 from 4079 g) bleed 4 from 4080 h) bleed 4 from 4081. As can be seen most of the bleeds produced good results, except for the bleeds from rabbit 4081.

FIG. 9: Western blot showing the specificity of the antibodies to the KHC coverstrand of D. melanogaster. Constructs that contain the wildtype coverstrand (WT, NL, L13, NL-L13) show targeting. The construct 2G also was targeted by the antibody, but this was expected as this construct has two residues mutated to glycine, so the majority of the coverstrand contains the wildtype residues, and the glycine residues should act to make the coverstrand more flexible, thus potentially conformally more amenable to detection.

FIG. 10 shows an antibody titration curve, which shows the disruption of Kinesin-1's motility as a function of the concentration of antibody. Only beads that tethered and did not run at all during the experiment were used for this analysis. The concentration of kinesin used was slightly above the single molecule limit. Fits to the data included a model that does not include coopertivity, but uses the pseudo-first order approximation, where antibody depletion is not accounted for (equation 2.1), a model that does account for antibody depletion, but not coopertivity (equation 2.2), and a model that includes the possibility of cooperativity (equation 2.3). The model allowing for coopertivity fit the data with the highest fidelity. The estimated equilibrium dissociation constant for the coopertivity model was in the low micromolar range with a coopertivity of nearly two.

### Example 2

### Tables

TABLE 2.1
Sequences of synthetic peptide used for
immunization of rabbits for polyclonal antibody
generation. These sequences correspond to parts
of Kinesin-1's coverstrand. Version 1 includes the
last six residues of the coverstrand plus two
glycine residues and cysteine. The cysteine was
added to conjugate the peptide to a substrate for
immunization, and the glycine residues were added
as flexible peptides. Version 2 contains all of the
residues of the Kinesin-1 coverstrand. As with
version 1, a C-terminal cysteine was
added for conjugation.

| | | | | | | | | | | | | | | | | |
|---|---|---|---|---|---|---|---|---|---|---|---|---|---|---|---|---|
| Version 1 | — | — | — | — | — | — | — | P | A | E | D | S | I | G | G | C |
| SEQ ID NO: 21 | | | | | | | | | | | | | | | | |
| Version 2 | M | S | A | E | R | E | I | P | A | E | D | S | I | C | — | — |
| SEQ ID NO: 22 | | | | | | | | | | | | | | | | |

TABLE 2.2
Immunization schedule of the four rabbits used for antibody
production. Rabbits were injected with combinations of the
peptides shown in Table 2.1 four times to illicit an immune
response and produce antibodies specific to these peptides.

| Rabbit | Jul. 22, 2009 | Aug. 12, 2009 | Sep. 02, 2009 | Sep. 23, 2009 |
|---|---|---|---|---|
| E4078 | 0.4 mg | 0.2 mg | 0.2 mg | 0.2 mg |
| E4079 | Version 1 + | Version 1 + | Version 1 + | Version 1 + |
| E4080 | 0.4 mg | 0.2 mg | 0.2 mg | 0.2 mg |
| E4081 | Version 2 | Version 2 | Version 2 | Version 2 |

TABLE 2.3
Bleed schedule for the production of antibodies. The rabbits
were bled four times, with bleed 0 was taken as a baseline.

| | | Sep. 14, | Sep. 16, | Oct. 05, | Oct. 07, |
|---|---|---|---|---|---|
| Rabbit | Jul. 21, 2009 | 2009 | 2009 | 2009 | 2009 |
| E4078 | 5 mL | 25 mL | 25 mL | 25 mL | 25 mL |
| E4079 | (Bleed 0) | (Bleed 1) | (Bleed 2) | (Bleed 3) | (Bleed 4) |
| E4080 | | | | | |
| E4081 | | | | | |

TABLE 2.4
Fit parameters from the antibody titration curve. The two
models that do not allow for coopertivity were nearly identical,
showing that antibody depletion effects were not significant
and the pseudo-first order approximation was valid. The
equilibrium dissociation constant found for these noncooperative
models was approximately 50 nM, which show very good
specificity. The coopertivity model fit to a dissociation constant
of about 1.6 μM, which shows considerably lower affinity.
The coopertivity was nearly 2, as could be expected for
kinesin, which has two coverstrands per molecule, and thus
two binding sites for the antibodies. The coopertivity model fit
the data the best. The low K_{D} could be due to the use
of polyclonal antibodies rather than monoclonal antibodies
which would be more specific.

| | K_{D} (nM) | n |
|---|---|---|
| Pseudo-first order approximation | 50.13 | N/A |
| Accounting for ligand depletion | 49.42 | N/A |
| Coopertivity model | 1593 | 1.9 |

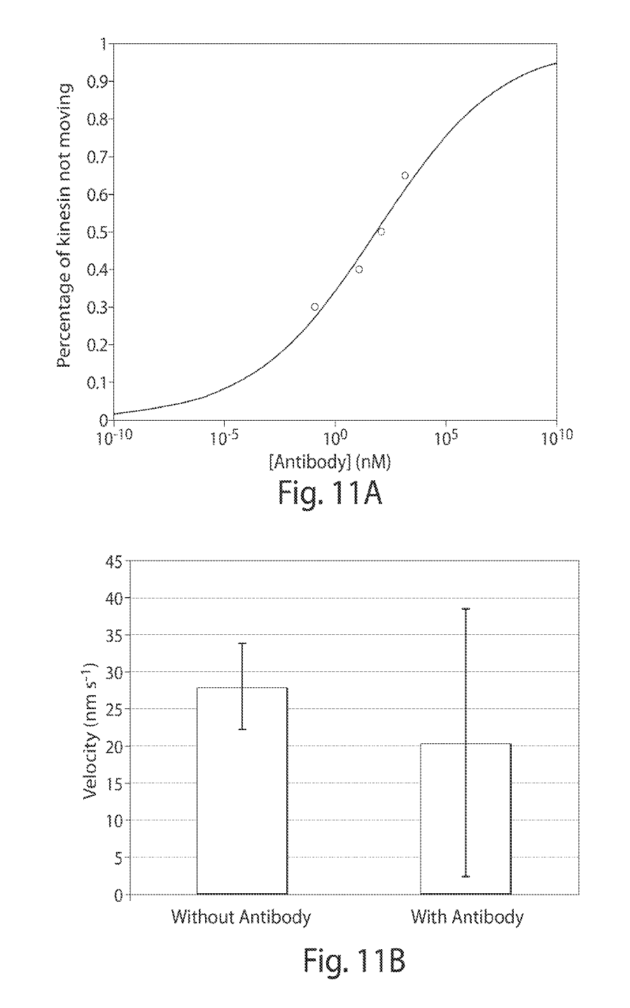

FIG. 11A shows an antibody titration curve produced with data from a kinesin motility assay, which shows the disruption of Kinesin-5's motility as a function of the concentration of a polyclonal antibody preparation comprising rabbit antibodies directed against amino acid sequence MASQPNSSAKKKEEKGKNI (SEQ ID NO: 23) of Human Eg5 (Kinesin-5) and MSAKKKEEKGKNI (SEQ ID NO: 17) of a chimeric kinesin. Only beads that tethered and did not run at all during the experiment were used for this analysis. The concentration of kinesin used was slightly above the single molecule limit.

### Example 3

### Protocols

### Kinesin Expression and Purification

### Materials

1. LB broth (with 100 μg/mL ampicillin+25 μg/mL chloramphenicol)
2. LB agar plates (with 100 m/mL ampicillin+25 μg/mL chloramphenicol)
3. TB broth, Add 47.6 g TB (Difco Terrific Broth) and 4 mL glycerol into IL deionized water and autoclave. Once cooled add ampicillin, chloramphenicol and biotin to final concentrations of 100 μm/mL, 25 μg/mL and 100 μM (24 mg), respectively.
4. 1M IPTG, prepared in water and stored at −20° C.
5. Rifampicin, prepared 20 mM (16.5 mg/mL) in methanol, 100× stock stored at −20° C.
6. Lysis buffer, 20 mM imidazole, 4 mM MgCl2, pH 7 (0.680 g imidazole, 0.408 mL 4.9M MgCl2 for 500 mL)
7. β-mercaptoethanol
8. PMSF, (Sigma-Aldrich P7626), 200 mM in isopropanol, stored at −20° C.
9. Pepstatin A, (Sigma-Aldrich P4265), 5 mg/mL in DMSO, stored at −20° C.
10. TPCK, (Sigma-Aldrich T4365), 10 mg/mL in DMSO, stored at −20° C.
11. TAME, (Sigma-Aldrich T4626), 40 mg/mL in deionized water, stored at −20° C.
12. Leupeptin, (Sigma-Aldrich L9875), 5 mg/mL in deionized water, stored at −20° C.

### 13. DNAse I (Sigma-Aldrich D4527), Grade II

### 14. RNAse A (Sigma-Aldrich RS000), Type II-A

### 15. Ni-NTA Resin, (Qiagen Ni-NTA Superflow)

16. TCEP, (Molecular Probes T-2566), 10 mM in deionized water, prepared fresh before use
17. Vivaspin 15 spin column, (Vivascience VS1522), 30,000 MWCO
18. Protease Inhibitor Cocktail, PI, prepare 4 mL of PI and store at −20° C. Contains: 160 μL 0.2 rng/rnL Pepstatin A, 800 μL 2 mg/mL TPCK, 200 μL. 2 mg/mL TAME, 160 μL 0.2 mg/rnL Leupeptin, 2 muL 2 mg/mL Soybean IT, 1880 μL deionized water

### 19. Econo-Column Chromatography Columns, (Bio-Rad 737-1512), 1.5×10 cm, 18 rnL

20. nuPAGE 4-12% Bis-Tris Gels, (Invitrogen NP0321BOX), 1 mm×10 well
21. Kinesin Storage Buffer, 50 mM imidazole. 100 mM NaCl2, 1 mM MgCl2, 20 μM ATP, 0.1 mM EDTA, 5% sucrose, pH 7

### Methods

Day 0

1. Streak fresh colonies on LB-agar plates containing ampicillin and chloramphenicol from frozen glycerol stocks stored at −80° C.

2. Incubate upside down (agar at the top) at 37° C. overnight

Day 1

1. Pick a single colony and add to 20 mL of LB (with ampicillin and thloramphenicol) in a 250 mL flask

2. Shake overnight at 37° C.

Day 2

1. Inoculate 500 mL of TB broth (with ampicillin and chloramplienicol) with 10 mL of the overnight LB culture

2. Shake at 37° C.

3. Induce expression at OD600=0.53−0.60 by adding IPTG to a final concentration of 1 mM

4. Upon induction, lower shaker temperature to 22° C. and shake overnight

Day 3

1. Centrifuge the cells at 5,000 g and 4° C. for 10 minutes

2. While centrifuging, add (3-mercaptoethanol (to a final concentration of 10 mM), 1/100 volume of PI, and 1/100 volume of PMSF to lysis buffer to make full lysis buffer. Make 5 mL for each lysis buffer

3. After centrifugation, drain the supernatant and resuspend the pellet in 5 mL of lysis buffer

4. Pipette the resuspended cells into a 15 mL centrifuge tube and incubate on ice for 30 minutes to allow the internal lysozyme to degrade the cell walls

5. Flash freeze the 15 ml tube in liquid nitrogen and store overnight at −80° C.

Day 4

1. Thaw frozen cells with alternating incubations in a 37° C. water bath and ice (1 minute in each, do not let the lysate warm up). Once completely thawed, flash freeze in liquid nitrogen. This process is repeated for a total of three thaws. After the last thaw, the lysate should be very viscous.

2. Add 500 μL 10 mg/mL RNAse (final concentration 1 mg/mL) and 250 μL 10 mg/mL DNAse (final concentration 0.5 mg/mL). Incubate on ice for 30 minutes with occasional mixing by inversion. The viscosity should substantially decrease

3. Centrifuge at 21,800 g and 4° C. for 20 minutes and retain the low speed supernatant. This step pellet out cellular debris

4. Centrifuge at 180,000 g and 4° C. for 30 minutes and retain the high speed supernatant

5. Add 2 mL of Ni-NTA equilibrated in full lysis buffer. To equilibrate Ni-NTA, wash the resin in full lysis buffer three times by centrifuging at 10,000 g and 4° C. for 10 minutes to pellet the resin. Remove supernatant and wash with full lysis buffer

6. Incubate the resin high speed supernatant mixture at 4° C. overnight with mixing by inversion

Day 5

1. Equilibrate the chromatography column by washing with 10 mL of full lysis buffer

2. Prepare 100 mL of elution buffer 1 and 2 (elution buffer 1 is the same as lysis buffer, but with β-mercaptoethanol, add 7 μL of β-mercaptoethanol to 100 mL of lysis buffer). To make elution buffer 2, add 3,268 g of imidazole to 100 mL of lysis buffer and adjust the pH to 7 with HCl, add 70 μL of β-mercaptoethanol

| Final [imidazole] | Elution Buffer 1 | Elution Buffer 2 |
|---|---|---|
| (mM) | (mL) | (mL) |
| 70 | 8.96 | 1.04 |
| 100 | 8.33 | 1.67 |
| 150 | 7.29 | 2.71 |
| 200 | 6.25 | 3.75 |
| 500 | 0 | 10 |

3. Load Ni-NTA high speed supernatant mixture into the column and collect the flow through

4. Wash five times with 10 mL of lysis buffer

5. After washing, run the imidazole gradient with increasing concentration of imidazole

6. Run samples from the flow through. washes, and gradient on an SDS-PAGE gel to determine which fractions contain kinesin and should be combined. Pool these fractions

7. Concentrate and buffer exchange the pooled fractions into kinesin storage buffer with a vivaspin concentrator, centrifuge at 4° C.

8. Aliquot the concentrated kinesin solution into 10 μL volumes and flash freeze in liquid nitrogen. Store at −80° C.

### Microtubule Polymerization

### Materials

1. PEM80, 80 rnM Pipes, 1 mM EGTA, 4 rnM MgCl₂, pH adjusted to 6.9 with KOH
2. PEM104, 103.6 mM Pipes, 13 mM EGTA 6.3 mM MgCl₂, pH adjusted to 6.9 with KOH
3. STAB, 34.1 μL, PEM80, 5 μL, 10 mM GTP stock (Cytoskeleton BST06), 47 μL, 60 g/L NaN₃, 1.2 μL 10 mM Taxol stock (Cytoskeleton TXDO1), 5 μM DSMO
4. Tubulin (Cytoskeleton T237 (now discontinued), replaced with TL238)

### Polymerization

1. Centrifuge tubulin aliquot at 10,000 g and 4° C. for 30 minutes
2. Combine 15.2 μL PEM104 and 2 μL 10 mM GTP to make PEM/GTP
3. Combine 15.2 μL PEM/GTP with 2.2 μL DMSO and vortex to mix. Add 4.8 μL of 10 mg/mL tubulin to make TUB
4. Place TUB in a water bath set to 37° C. for 30 minutes
5. Remove TUB from the water bath and add 2 μL of STAB
6. Store polymerized microtubules at room temperature, NOTE: reconstituted tubulin looses its polymerization ability after about a month at −80° C.

### Coverslide Preparation

### Materials

1. KOH pellets
2. 200 proof ethanol
3. Deionized water
4. Poly-1-lysine solution (Sigma-Aldrich P8920)

### KOH Etching

1. Add 100 g of KOH pellets to 300 mL of ethanol (in a 1,000 rnL beaker), use a magnetic stir bar to mix
2. Fill two 1000 mL beakers with 300 mL deionized water and one with 300 mL of ethanol
3. Using a bath sonicator, degas all of the solutions for five minutes
4. Place coverslips in teflon racks and immerse one rack at a time in the KOH solution sonicate for five minutes
5. Rinse etch slide in one of the beakers of ethanol, then in deionized water
6. Sonicate the rinsed slides in deionized water
7. Rinse slides with deionized water in using a squeeze bottle
8. Rinse slides with ethanol in using a squeeze bottle
9. Dry slides in an oven for 30 minutes

### Poly-Lysine Coating

1. Add 1 mL of poly-1-lysine solution to 300 mL of ethanol
2. Place a rack of KOH etched coverslides into the poly-lysine ethanol solution
3. Incubate at room temperature for 15 minutes
4. Dry in an oven for 15 minutes

### Kinesin Motility Assay

### Materials

### 1. PEM80

2. Phosphate buffered saline (PBS)
3. Taxol stock (10 mM in DMSO, stored at −20° C.)
4. DTT, 0.5M in 10 mM potassium acetate (stored at −20° C.)
5. ATP (100 mM in PEM80, stored at −80° C.)
6. Potassium acetate (3M, stored at 4° C.)
7. Casein (10 mg/mL in PBS with 0.1% tween 20 (PBST), made fresh the day of the experiment, filtered using a vacuum filter)
8. Kinesin stock (stored at −80° C.)
9. Streptavidin coated beads
10. Poly-lysine coated KOH etched coverslides
11. Double sided sticky tape
12. Glucose oxidase (100× stock (Calbiochem 345386), 25 mg/mL in PBST, stored at −80° C.)
13. β-D-glucose (100× stock, 500 mg/mL in PBST, stored at −80° C. 14. Catalase (100× stock (Calbiochem 219261), 3 mg/mL in PBST, stored at −80° C.)

### Assay Preparation

1. Make PemTax: Add 1,000 μL PEM80 and 2 μL Taxol, store at room temperature
2. Make assay buffer (AB, final concentrations 0.1 mM DTT, 20 μM Taxol, 0.2 mg/mL Casein, 1 mM ATP, 50 mM potassium acetate), store on ice
(a) 2,848 μL PEM80
(b) 6 μL 0.5M DTT
(c) 6 μL 10 mM Taxol
(d) 30 μL 100 mM ATP
(e) 50 μL 3M potassium acetate
(f) 60 μL 10 mg/mL casein
   3. Make C-Tax: 80 μL PemTax and 20 μL 10 mg/mL casein, store on ice
   4. Made bead dilutions
(a) Dilute 20 μL of 0.44 μm streptavidin coated beads into 80 μL of PBS
(b) Wash beads five times with PBS by centrifuging at 10,000 RPM for 6 minutes, discarding the supernatant and resuspending in 100 μL of PBS
(c) Sonicate the beads twice for two minutes using a cup horn sonicator filled with water and ice
(d) Make EM/AB by adding 8 μL of washed and sonicated beads to 392 μL of AB, store on ice
   5. Make kinesin dilutions
(a) K/100: 2 μL kinesin stock into 98 μL AB (note, this is actually twice as concentrated as the label suggests, but will be come correct upon adding beads in subsequent steps)
(b) K/1000: 10 μL, of K/100 into 90 μL AB
(c) Continue diluting in this manner until experiments show that half or less of the beads move at a given dilution, sometimes K/10⁷⁻⁸ were necessary. Store all dilutions on ice
   6. Make Kinesin+Bead dilutions (KDB/###)
(a) KDB/100: 50 μL EM/AB added to 50 μL K/100
(b) Continue making these dilutions for the kinesin concentrations to be tested
(c) Incubate for 1 hour at 4° C.
   7. Make MT/150 by adding 1 μL of polymerized microtubules to 149 μL PernTax, do NOT place on ice
   8. Make flow cells for the assay
(a) Place two pieces of double sided tape perpendicular to the long axis of a thick glass slide
(b) Place a poly-lysine coated coverslide on top of the tape to make a chamber
(c) Flow 15 μL of MT/150 and allow to incubate at room temperature for 10 minutes
(d) Wash the chamber with 20 μL of PemTax
(e) Flow in 15 μL of C-Tax and incubate at room temperature for 5 minutes
(f) Wash with 50 μL of PernTax followed by 80 μL of AB
(g) Flow in 20 μL of the KDB dilution to be assayed

### Stall Force Assay

1. Turn on the trapping and detection lasers
2. Turn on the AOD amplifier
3. Load the flow cell slide containing the kinesin sample to be assayed
4. Turn on monitors and camera controller
5. Make adjustments with the microscope (focus, condenser height, filter position) to image the microtubules and beads
6. Unblock the trapping laser
7. Run the VI to initialize the AODs
8. Test beads for movement
(a) Trap a diffusing bead
(b) Hold the bead near a microtubule for a few seconds, if it moves, go on, if not, then try the bead on two different microtubules to test for motility
   i. For moving beads, run AOD line sweep which is used to align the AODs with the position detection system. Adjust the micrometers for the detection branch to bring the AODs and detection branch into alignment
   ii. Once aligned, run the calibration VI, which sets the calibration for conversion between QPD voltage and nanometer space, as well as running the calibration for trap stiffness, which uses the variance method
   iii. After calibration, start the VI to record the voltage signals from the VI
   iv. Place the bead near a microtubule, as before, and record the movement of the bead assay, it is just to ensure that the voltage signal used to actuate the trapping laser's shutter is not anomalous

### Unloaded Assay

1. Turn on the trapping and detection lasers
2. Turn on the AOD amplifier
3. Load the flow cell slide containing the kinesin sample to be assayed
4. Turn on monitors and camera controller
5. Make adjustments with the microscope (focus. condenser height, filter position) to image the microtubules and beads
6. Unblock the trapping laser
7. Run the VI to initialize the AODs
8. Test beads for movement
(a) Trap a diffusing bead
(b) Hold the bead near a microtubule for a few seconds, if it moves, go on, if not, then try the bead on two different microtubules to test for motility
   i. For moving beads, run AOD line sweep which is used to align the AODs with the position detection system. Adjust the micrometers for the detection branch to bring the AODs and detection branch into alignment. This does not have to be as exact as with the stall force
   ii. Place the bead near a microtubule, as before, and run the Vito shutter the trapping laser after movement of kinesin, the switch that switches between foot switch actuation and computer actuation needs to be switched to computer control
   iii. Once the bead begins to run, the trapping laser will be shuttered and the bead will be free to walk on the microtubule. After the bead dissociates from the microtubule, un-shutter the trapping laser using the VI and try to recapture the bead for further runs

### Gliding Filament Assay

A gliding filament assay was performed to evaluate binding of a polyclonal antibody preparation directed against amino acid sequence MASQPNSSAKKKEEKGKNI (SEQ ID NO: 23) of Human Eg5 (Kinesin-5). The assay was performed according to methods known in the art. See, e.g., Weinger et al, “A Nonmotor Microtubule Binding Site in Kinesin-5 Is Required for Filament Crosslinking and Sliding” Curr. Biol. (2011) 21, 154-160. Briefly, the assay involved adsorbing histidine tagged full length Human Eg5 (Kinesin-5) to a glass coverslide. Next, in separate containers microtubules were contacted with the adsorbed Kinesin-5 in the presence (at 2 mg/mL) or absence of the antibody preparation. The assay was run 3 times with antibody and 2 times without. The velocity of the microtubules was quantified in both cases. The velocity of the microtubules without antibody is comparable to that reported in the literature. See, e.g., Weinger et al, “A Nonmotor Microtubule Binding Site in Kinesin-5 Is Required for Filament Crosslinking and Sliding” Curr. Biol. (2011) 21, 154-160. A decrease in velocity, and increase in standard deviation of the microtubules' speed, was observed in the presence of the antibody preparation, which is indicative of the antibody acting to inhibit motor action. These results are depicted in FIG. 11B.

### REFERENCES

[1] M D Wang, M J Schnitzer, H Yin, R Landick, J Genes, and S M Block. Force and velocity measured for single molecules of ma polymerase. Science, 282(5390):902, 1998.
[2] Dennis Huszar, Maria-Elena Theoclitou, Jeffrey Skolnik, and Ronald Herbst. Kinesin motor proteins as targets for cancer therapy. Cancer Metastasis Rev, 28(1-2):197-208, June 2009.
[3] W Hwang, M J Lang, and M Karplus. Force generation in kinesin hinges on cover-neck bundle formation. Structure, 16(1):62-71, 2008.
[4] K Svoboda and S M Block. Force and velocity measured for single kinesin molecules. Cell, 77(5):773-84, June 1994.
[5] Steven M Block. Kinesin motor mechanics: Binding, stepping, tracking, gating, and limping. Biophysical Journal, 92(9):2986-2995, January 2007.
[6] Ahmed S Khalil, David C Appleyard, Anna K Labno, Adrien Georges, Martin Karplus, Angela M Belcher, Wonmuk Hwang, and Matthew J Lang. Kinesin's cover-neck bundle folds forward to generate force. Proc Nail Acad Sci USA, 105(49):19247-52, December 2008.
[7] S Rice, A W Lin, D Safer, C L Hart, N Naber, B Carragher, S M Cain, E Pechatnikova, E M Wilson-Kubalek, M Whittaker, E Pate, R Cooke, E W Taylor, R A Milligan, and R D Vale. A structural change in the kinesin motor protein that drives motility. Nature, 402(6763):778-84, December 1999.
[8] S Rice, Y Cui, C Sindelar, N Naber, M Matuska, R Vale, and ft Cooke. Thermodynamic properties of the kinesin neck-region docking to the catalytic core. Biophysical Journal, 84(3):1844-1854, December 2003.
[9] Nikolina Kalchishkova and Konrad J Bohm. The role of kinesin neck linker and neck in velocity regulation. Journal of Molecular Biology, 382(1):127-35, September 2008.
[10] Nikolina Kalchishkova and Konrad J Bohm. On the relevance of the core helix alpha 6 to kinesin activity generation. Biophysical Reviews and Letters, 4(1 & 2):63-75, June 2009.
[11] Stefan Lakiimper, Christina Thiede, Andre Diiselder, Stefanie Reiter, Mikhail J Korneev, Lukas C Kapitein, Erwin J G Peterman, and Christoph F Schmidt. The effect of monastrol on the processive motility of a dimeric Kinesin-5 head/kinesin 1 stalk chimera. Journal of Molecular Biology, 399(1):1-8, May 2010.
[12] C Thiede, S Lakiimper, A D Weilel, S Reiter, and C F Schmidt. Tetrarneric chimera dk4rner is a tool to study mechanisms of Kinesin-5 regulation. a tetrameric chimera of a Kinesin-1 and a Kinesin-5 is a fast microtubule motor. Biophysical Journal, 98(3):165a, January 2010.
[13] C V Sindelar, M J Budny, S Rice, N Naber, R Fletterick, and R Cooke. Two conformations in the human kinesin power stroke defined by x-ray crystallography and epr spectroscopy. Nature Structural f.4 Molecular Biology, 9(11):844-848, 2002.
[14] Andrew J Bodey, Masahide Kikkawa, and Carolyn A Moores. 9-angstrom structure of a microtubule-bound mitotic motor. J Mol Biol, 388(2):218-224, January 2009.
[15] Adam G Larson, Nariman Naber, Roger Cooke, Edward Pate, and Sarah E Rice. The conserved 15 loop establishes the pre-powerstroke conformation of the Kinesin-5 motor, eg5. Biophysj, 98(11):2619-2627, February 2010.
[16] Z Maliga, T M Kapoor, and T J Mitchison. Evidence that monastrol is an allosteric inhibitor of the mitotic kinesin eg5. Chemistry el biology, 9(9):989-996, 2002.
[17] Mikhail J Korneev, Stefan Lakamper, and Christoph F Schmidt. Load-dependent release limits the processive stepping of the tetrameric eg5 motor. Eur Biophys J, 36(6):675-81, July 2007.
[18] Megan T Valentine, Polly M Fordyce, Troy C Krzysiak, Susan P Gilbert, and Steven M Block. Individual dimers of the mitotic kinesin motor eg5 step processively and support substantial loads in vitro. Nature Cell Biology, 8(5):470-476, May 2006.
[19] E Berliner, E C Young, K Anderson, H K Mahtani, and J Gelles. Failure of a single-headed kinesin to track parallel to microtubule protofilaments. Nature, 373(6516):718-21, February 1995.
[20] M E Fisher and A B Kolomeisky. The force exerted by a molecular motor. Proc Natl Acad Sri USA, 96(12):6597-602, June 1999.
[21] M E Fisher and A B Kolomeisky. Simple mechanochemistry describes the dynamics of kinesin molecules. Proc Natl Acad Sci USA, 98(14):7748-53, July 2001.
[22] S M Block, C L Asbury, J W Shaevitz, and M J Lang Probing the kinesin reaction cycle with a 2d optical force clamp. Proceedings of the National Academy of Sciences, 100(5):2351-2356, 2003.
[23] C M Coppin, D W Pierce, L Hsu, and R D Vale. The load dependence of kinesin's mechanical cycle. Proc Natl Acad Sci USA, 94(16):8539-44, August 1997.
[24] M J Schnitzer, K Visscher, and S M Block. Force production by single kinesin motors. Nature Cell Biology, 2(10):718-23, October 2000.
[25] Ryo Nitta, Yasushi Okada, and Nobutaka Hirokawa. Structural model for strain-dependent microtubule activation of mg-ADP release from kinesin. Nat Struct Mol Biol, 15(10):1067-75, October 2008.
[26] R B Case, S Rice, C L Hart, B Ly, and R D Vale. Role of the kinesin neck linker and catalytic core in microtubule-based motility. Curr Biol, 10(3):157-60, February 2000.
[27] Kuniyoshi Kaseda, Hideo Higuchi, and Keiko Hirose. Coordination of kinesin's two heads studied with mutant heterodimers. Proc Natl Acad Sci USA, 99(25):16058-63, December 2002.
[28] M E Aubin-Tam, D C Appleyard, E Ferrari, V Garbin, 00 Fadiran, J Kunkel, and M J Lang. Adhesion through single peptide aptamers. The Journal of Physical Chemistry A, page 2672, 2011.
[29] K. M. R Bhat and V Setaluri. Microtubule-associated proteins as targets in cancer chemotherapy. Clinical Cancer Research, 13(10):2849-2854, May 2007.
[30] Kuan Yoow Chan, Keith R Matthews, and Klaus Ersfeld. Functional characterisation and drug target validation of a mitotic Kinesin-13 in trypanosoma brucei. PLoS Pathoy, 6(8):e1001050, August 2010.
[31] S DeBorns, D A Skoufias, L Lebeau, 11 Lopez, G Robin, R L Margolis, R E Wade, and F Kozielslci. In vitro screening for inhibitors of the human mitotic kinesin eg5 with antimitotic and antitumor activities. Molecular cancer therapeutics, 3(9):1079, 2004.
[32] E Koller, S Propp, H Zhang, C Zhao, X Xiao, M Y Chang, S A Hirsch, P J Shepard, S Koo, and C Murphy. Use of a chemically modified antisense oligonucleotide library to identify and validate eg5 (kinesin-like 1) as a target for antineoplastic drug development. Cancer Research, 66(4):2059, 2006.
[33] Frank Kozielski, Dimitrios A Skoufias, Rose-Laure Indorato, Yasrnina Saoudi, Peter R Jungblut, Hanne K Hustoft, Margarita Strozynski, and Bernd Thiede. Proteome analysis of apoptosis signaling bys-trityl-1-cysteine, a potent reversible inhibitor of human mitotic kinesin eg5. Proteomics, 8(2):289-300, January 2008.
[34] R Nakai, S.-I lida, T Takahashi, T Tsujita, S Okamoto, C Takada, K Akasaka, S Ichikawa, H Ishida, H Kusaka, S Akinaga, C Murakata, S Honda, M Nitta, H Saya, and Y Yamashita. K858, a novel inhibitor of mitotic Kinesin Eg5 and antitumor agent, induces cell death in cancer cells. Cancer Research, 69(9):3901-3909, May 2009.
[35] Ole Henrik Brekke and Inger Sandlie. Therapeutic antibodies for human diseases at the dawn of the twenty-first century. Nat Rev Drug Discov, 2(1):52-62, January 2003.
[36] M Harris. Monoclonal antibodies as therapeutic agents for cancer. The lancet oncology, 5(5):292-302, 2004.
[37] S T Brady, K K Pfister, and G S Bloom. A monoclonal antibody against Kinesin-Inhibits both anterograde and retrograde fast axonal transport in squid axoplasm. Proc Natl Aced Sci USA, 87(3):1061-5, February 1990.
[38] A L Ingold, S A Cohn, and J M Scholey. Inhibition of kinesin-driven microtubule motility by monoclonal antibodies to kinesin heavy chains. The Journal of Cell Biology, 107(6 Pt 2):2657-67, December 1988.
[39] V A Lombillo, C Nislow, T J Yen, V I Geifand, and J R McIntosh. Antibodies to the kinesin motor domain and CENP-e inhibit microtubule depolymerization dependent motion of chromosomes in vitro. The Journal of Cell Biology, 128(12):107-15, January 1995.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A chimeric kinesin protein comprising:
one or more regions having an amino acid sequence of a kinesin protein that is not a Kinesin-5 protein; and
(i.) a coverstrand having an amino acid sequence of a coverstrand of a Kinesin-5 protein; or
(ii.) a necklinker having an amino acid sequence of a necklinker of a Kinesin-5 protein; or
(iii.) an L13 region having an amino acid sequence of an L13 region of a Kinesin-5 protein.

2. The chimeric kinesin protein of claim 1, comprising a coverstrand having an amino acid sequence of a coverstrand of a Kinesin-5 protein.

3. The chimeric kinesin protein of claim 1, comprising a coverstrand having the complete amino acid sequence of a coverstrand of a Kinesin-5 protein.

4. The chimeric kinesin protein of claim 1, comprising a necklinker having an amino acid sequence of a necklinker of a Kinesin-5 protein, optionally wherein the amino acid sequence of the necklinker of the Kinesin-5 protein is of the β9 region of the necklinker.

5. The chimeric kinesin protein of claim 1, comprising a necklinker having the complete amino acid sequence of a necklinker of a Kinesin-5 protein.

6. The chimeric kinesin protein of claim 1, comprising an L13 region having an amino acid sequence of an L13 region of a Kinesin-5 protein.

7. The chimeric kinesin protein of claim 1, comprising an L13 region having the complete amino acid sequence of an L13 region of a Kinesin-5 protein.

8. The chimeric kinesin protein of claim 1, wherein the Kinesin-5 protein is a human Kinesin-5 protein.

9. The chimeric kinesin protein of claim 1, wherein the Kinesin-5 protein is a Kinesin-5 protein of a species selected from the group consisting of: Arabidopsis thaliana; Aspergillus nidulans; Bombyx mori; Candida albicans; Caenorhabditis elegans; Chlamydomonas rheinhardtii; Cricetulus griseus; Cyanophora paradoxa; Cylindrotheca fusiformis; Danio rerio; Dictyostelium discoideum; Drosophila melanogaster; Drosophila yakuba; Gallus gallus; Homo Sapiens; Leishmania chagasi; Leishmania major; Loligo pealii; Lymantria dispar; Monodelphis domestica; Morone saxatilis; Mus musculus; Nectria haematococca; Neurospora crassa; Nicotiana tabacum; Oryza sativa; Paracentrotus lividus; Plasmodium falciparum; Rattus norvegicus; Saccharomyces cerevisiae; Schizosaccharomyces pombe; Solanum tuberosum; Strongylocentrotus purpuratus; Syncephalastrum racemosum; Tetrahymena thermophila; Trypanosoma brucei; Ustilago maydis; Volvox carteri; and Xenopus laevis.

10. The chimeric kinesin protein of claim 1, wherein the kinesin protein that is not a Kinesin-5 protein is selected from the group consisting of: Kinesin-1, Kinesin-2, Kinesin-3, Kinesin-4, Kinesin-6, Kinesin-7, Kinesin-8, Kinesin-9, Kinesin-10, Kinesin-11, Kinesin-12, Kinesin-13, and Kinesin-14.

11. The chimeric kinesin protein of claim 1, wherein the kinesin protein that is not a Kinesin-5 protein is Kinesin-1.

12. The chimeric kinesin protein of claim 1, wherein the kinesin protein that is not a Kinesin-5 protein a non-human kinesin protein.

13. The chimeric kinesin protein of claim 12, wherein the non-human kinesin protein is a kinesin protein of a species selected from the group consisting of: Arabidopsis thaliana; Aspergillus nidulans; Bombyx mori; Candida albicans; Caenorhabditis elegans; Chlamydomonas rheinhardtii; Cricetulus griseus; Cyanophora paradoxa; Cylindrotheca fusiformis; Danio rerio; Dictyostelium discoideum; Drosophila melanogaster; Drosophila yakuba; Gallus gallus; Leishmania chagasi; Leishmania major; Loligo pealii; Lymantria dispar; Monodelphis domestica; Morone saxatilis; Mus musculus; Nectria haematococca; Neurospora crassa; Nicotiana tabacum; Oryza sativa; Paracentrotus lividus; Plasmodium falciparum; Rattus norvegicus; Saccharomyces cerevisiae; Schizosaccharomyces pombe; Solanum tuberosum; Strongylocentrotus purpuratus; Syncephalastrum racemosum; Tetrahymena thermophila; Trypanosoma brucei; Ustilago maydis; Volvox carteri; and Xenopus laevis.

14-22. (canceled)

23. A chimeric kinesin protein comprising:
one or more regions having an amino acid sequence of a first kinesin protein; and
(i.) a coverstrand having an amino acid sequence of a coverstrand of second kinesin protein; or
(ii.) a necklinker having an amino acid sequence of a necklinker of a second kinesin protein; or
(iii.) an L13 region having an amino acid sequence of an L13 region of a second kinesin protein, wherein the first kinesin protein is different than the second kinesin protein.

24. The chimeric kinesin protein of claim 23, wherein the first kinesin protein and second kinesin protein are each selected from the group consisting of Kinesin-1, Kinesin-2, Kinesin-3, Kinesin-4, Kinesin-5, Kinesin-6, Kinesin-7, Kinesin-8, Kinesin-9, Kinesin-10, Kinesin-11, Kinesin-12, Kinesin-13, and Kinesin-14 proteins.

25. A chimeric kinesin protein comprising an amino acid sequence as set forth in any of SEQ ID NO: 3-7.

26-33. (canceled)

34. A method for characterizing the ability of a test agent to affect motility of a kinesin protein, the method comprising
(i.) obtaining a chimeric kinesin protein comprising one or more regions having an amino acid sequence of a first kinesin protein; and one or more regions having an amino acid sequence of a second kinesin protein selected from the group consisting of:
(1) a coverstrand having an amino acid sequence of a coverstrand of second kinesin protein;
(2) a necklinker having an amino acid sequence of a necklinker of a second kinesin protein; and
(3) an L13 region having an amino acid sequence of an L13 region of a second kinesin protein,
wherein the first kinesin protein is different than the second kinesin protein; and
(ii.) assessing motility of the chimeric kinesin protein in the presence the test agent.

35. The method of claim 34, wherein step (ii.) comprises:
(a.) subjecting the chimeric kinesin protein to a motility assay in the presence the test agent, wherein the results of the motility assay indicate whether the test agent inhibits motility of the chimeric kinesin protein;
(b.) subjecting the first kinesin protein to a motility assay in the presence the test agent, wherein the results of the motility assay indicate whether the test agent inhibits motility of the first kinesin protein; and
(c) comparing the results of the motility assay in (a.) with the results of the motility assay in (b.),
wherein if the test agent inhibits motility of the chimeric kinesin protein but does not substantially inhibit motility of the first kinesin protein, then the test agent is identified as targeting the one or more regions of the second kinesin protein.

36. The method of claim 34, wherein step (ii.) comprises:
(a.) subjecting the chimeric kinesin protein to a motility assay in the presence the test agent, wherein the results of the motility assay indicate whether the test agent inhibits motility of the chimeric kinesin protein;
(b.) comparing the results of the motility assay in (a.) with the results of a motility assay indicative of whether the test agent inhibits motility of the first kinesin protein,
wherein if the test agent inhibits motility of the chimeric kinesin protein but does not substantially inhibit motility of the first kinesin protein, then the test agent is identified as targeting the one or more regions of the second kinesin protein.

37. The method of claim 35, wherein the motility assay is a gliding filament assay or a stall force assay.
